(19) United States
(12) Patent Application Publication
(10) Pub. No.: US 2023/0131150 A1
(43) Pub. Date: Apr. 27, 2023

(54) UNCONSCIOUSNESS ESTIMATION APPARATUS, UNCONSCIOUSNESS ESTIMATION METHOD AND PROGRAM

(71) Applicant: NIPPON TELEGRAPH AND TELEPHONE CORPORATION, Tokyo (JP)

(72) Inventors: Shingo TSUKADA, Musashino-shi, Tokyo (JP); Hiroshi NAKASHIMA, Musashino-shi, Tokyo (JP); Masumi YAMAGUCHI, Musashino-shi, Tokyo (JP); Kentaro TANAKA, Musashino-shi, Tokyo (JP); Toichiro GOTO, Musashino-shi, Tokyo (JP)

(73) Assignee: NIPPON TELEGRAPH AND TELEPHONE CORPORATION, Tokyo (JP)

(21) Appl. No.: 17/912,174

(22) PCT Filed: Mar. 19, 2020
(86) PCT No.: PCT/JP2020/012440

§ 371 Date: Sep. 16, 2022

(51) Int. Cl. A61B 5/00; A61B 5/026; A61B 5/363

(52) U.S. Cl. CPC A61B 5/4809; A61B 5/026; A61B 5/7221; A61B 5/363; A61B 5/7275

(57) ABSTRACT

An aspect of the present invention is a loss-of-consciousness estimation apparatus including: a ventricular state estimation unit configured to estimate whether or not a ventricular state of an estimation target is normal in a predetermined repetition cycle, based on a cerebral blood flow correlation amount time series, which is a time series of an amount correlated with a cerebral blood flow rate of the estimation target; a measurement reliability estimation unit configured to estimate reliability of the cerebral blood flow correlation amount time series; and a loss-of-consciousness probability acquisition unit configured to, based on the reliability, the estimation result of the ventricular state estimation unit, and elapsed time after a probability acquisition start time, which is a time at which it is first determined by the ventricular state estimation unit that the ventricular state is not normal after an estimation start time, which is a time of starting the repetition cycle, acquire a loss-of-consciousness probability indicating a probability that the estimation target has already lost consciousness.

### TECHNICAL FIELD

The present invention relates to a loss-of-consciousness estimation apparatus, a loss-of-consciousness estimation method, and a program.

### BACKGROUND ART

Unintentional loss of consciousness during work is dangerous for both the person experiencing it and those around the person. For example, if a driver loses consciousness while driving, passengers including the driver and the people in the surrounding area of the car are at risk (NPL 1).

### CITATION LIST

### Non-Patent Literature

[NPL 1] Kazuaki Shinohara, Tomomi Komaba, Katsuhiko Hashimoto, Fumito Ito, Megumi Okada, Tokiya Ishida, Hideyuki Yokoyama, Akinori Matsumoto, “Examination of Cases of Consciousness Disorder Attacks While Driving”, Transactions of the Society of Automotive Engineers of Japan/Volume 45 (2014) No. 6, p. 1105-1110

### SUMMARY OF THE INVENTION

### Technical Problem

If the person and people around the person can find out that the likelihood of loss of consciousness is high before such a loss of consciousness (hereinafter referred to as “loss of consciousness”) occurs, the danger posed by the loss of consciousness can be reduced. For this reason, it is required that the accuracy of estimating the likelihood of loss of consciousness is improved.

In view of the above circumstances, it is an object of the present invention to provide a technique for improving the accuracy of estimating the likelihood of loss of consciousness.

### Means for Solving the Problem

An aspect of the present invention is a loss-of-consciousness estimation apparatus including: a ventricular state estimation unit configured to estimate whether or not a ventricular state of an estimation target is normal in a predetermined repetition cycle, based on a cerebral blood flow correlation amount time series, which is a time series of an amount correlated with a cerebral blood flow rate of the estimation target; a measurement reliability estimation unit configured to estimate reliability of the cerebral blood flow correlation amount time series; and a loss-of-consciousness probability acquisition unit configured to, based on the reliability, the estimation result of the ventricular state estimation unit, and elapsed time after a probability acquisition start time, which is a time at which it is first determined by the ventricular state estimation unit that the ventricular state is not normal after an estimation start time, which is a time of starting the repetition cycle, acquire a loss-of-consciousness probability indicating a probability that the estimation target has already lost consciousness.

### Effects of the Invention

According to the present invention, it is possible to improve the accuracy of estimating the likelihood of loss of consciousness.

### BRIEF DESCRIPTION OF DRAWINGS

FIG. 1 is an explanatory diagram illustrating an overview of a loss-of-consciousness estimation system 100 of a first embodiment.

FIG. 2 is a diagram showing an upper threshold value, a lower threshold value, a threshold value region, and out-of-range data in the first embodiment.

FIG. 3 is a diagram showing an example of a system configuration of the loss-of-consciousness estimation system 100 of the first embodiment.

FIG. 4 is a diagram showing an example of a functional configuration of a control unit 20 in the first embodiment.

FIG. 5 is a flowchart showing an example of a flow of processing executed by the loss-of-consciousness estimation system 100 in the first embodiment.

FIG. 6 is a first explanatory diagram illustrating a relationship between a loss-of-consciousness probability and a cerebral blood flow correlation amount time series in the first embodiment.

FIG. 7 is a second explanatory diagram illustrating the relationship between the loss-of-consciousness probability and the cerebral blood flow correlation amount time series in the first embodiment.

FIG. 8 is a third explanatory diagram illustrating the relationship between the loss-of-consciousness probability and the cerebral blood flow correlation amount time series in the first embodiment.

FIG. 9 is a fourth explanatory diagram illustrating the relationship between the loss-of-consciousness probability and the cerebral blood flow correlation amount time series in the first embodiment.

FIG. 10 is a diagram showing an example of a system configuration of a loss-of-consciousness estimation system 100a of a second embodiment.

FIG. 11 is a diagram showing an example of a functional configuration of a control unit 20a in the second embodiment.

FIG. 12 is a first flowchart showing an example of a flow of processing executed by the loss-of-consciousness estimation system 100a of the second embodiment.

FIG. 13 is a second flowchart showing an example of a flow of processing executed by the loss-of-consciousness estimation system 100a of the second embodiment.

FIG. 14 is a diagram showing an example of a system configuration of a loss-of-consciousness estimation system 100b of a third embodiment.

FIG. 15 is a diagram showing an example of a functional configuration of the control unit 20b according to the third embodiment.

FIG. 16 is a diagram showing an example of a system configuration of a loss-of-consciousness estimation system 100c of a fourth embodiment.

FIG. 17 is a diagram showing an example of a functional configuration of a control unit 20c according to the fourth embodiment.

FIG. 18 is a diagram showing an example of a system configuration of a loss-of-consciousness estimation system 100d according to a fifth embodiment.

FIG. 19 is a diagram showing an example of a functional configuration of a control unit 20d according to the fifth embodiment.

FIG. 20 is a diagram showing an example of a system configuration of a loss-of-consciousness estimation system 100e according to a sixth embodiment.

FIG. 21 is a diagram showing an example of a functional configuration of a control unit 20e according to the sixth embodiment.

FIG. 22 is a diagram showing an example of a cardiac potential time series before shaping in a second variation.

FIG. 23 is a diagram showing an example of a cardiac potential time series after shaping through high-pass filter processing in the second variation.

FIG. 24 is a diagram showing an example of a functional configuration of a control unit 20f in the second variation.

FIG. 25 is a first diagram showing a correspondence between a cardiac potential time series and a model waveform in a third variation.

FIG. 26 is a second diagram showing a correspondence between the cardiac potential time series and the model waveform in the third variation.

FIG. 27 is a third diagram showing the correspondence between the cardiac potential time series and the model waveform in the third variation.

FIG. 28 is a fourth diagram showing the correspondence between the cardiac potential time series and the model waveform in the third variation.

FIG. 29 is a first diagram showing the correspondence between an aortic blood flow rate time series and the model waveform in the third variation.

FIG. 30 is a second diagram showing the correspondence between the aortic blood flow rate time series and the model waveform in the third variation.

FIG. 31 is a diagram showing an example of a functional configuration of a control unit 20g in the third variation.

### DESCRIPTION OF EMBODIMENTS

### First Embodiment

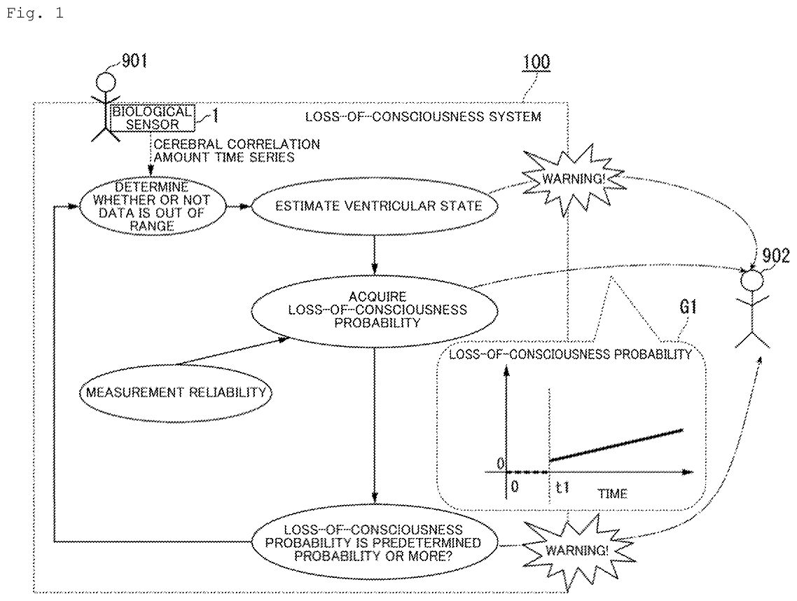

FIG. 1 is an explanatory diagram illustrating an overview of a loss-of-consciousness estimation system 100 of a first embodiment. The loss-of-consciousness estimation system 100 estimates the probability that an estimation target 901 has already lost consciousness. The estimation target 901 may be a person or an animal. Hereinafter, for the sake of simplicity, the loss-of-consciousness estimation system 100 will be described taking, as an example, a case where the estimation target 901 is a person.

The loss-of-consciousness estimation system 100 includes a biological sensor 1. The biological sensor 1 measures an amount (hereinafter referred to as “cerebral blood flow correlation amount”) that is correlated with the cerebral blood flow rate of the estimation target 901 to be measured. The loss-of-consciousness estimation system 100 acquires a time series of the cerebral blood flow correlation amount of the estimation target 901 (hereinafter referred to as “cerebral blood flow correlation amount time series”) using the biological sensor 1. Details of the biological sensor 1 will be described later.

The cerebral blood flow correlation amount may be any amount as long as it is an amount that is correlated with the cerebral blood flow rate of the estimation target 901. The cerebral blood flow correlation amount may be, for example, an amount indicating the state of electrical activity of the heart. An amount indicating the state of electrical activity of the heart is, for example, the change over time in the electrical potential (cardiac potential) indicated by a graph of an electrocardiogram. That is, the amount indicating the state of electrical activity of the heart is a time series of the cardiac potential (hereinafter referred to as “cardiac potential time series”). In such a case, the cerebral blood flow correlation amount time series is a graph of an electrocardiogram.

The cerebral blood flow correlation amount may be, for example, the blood flow rate of the carotid artery or the aorta. In such a case, the cerebral blood flow correlation amount time series is the time series of the aortic blood flow rate. Hereinafter, for the sake of simplicity in the description, the loss-of-consciousness estimation system 100 will be described taking, as an example, a case where the cerebral blood flow correlation amount time series is a cardiac potential time series.

The loss-of-consciousness estimation system 100 starts acquiring the cerebral blood flow correlation amount time series of the estimation target 901 at a predetermined timing, and thereafter repeatedly acquires the cerebral blood flow correlation amount time series of the estimation target 901 in a predetermined repetition cycle (hereinafter referred to as an “estimation cycle”). Hereinafter, the time when the processing for repeatedly acquiring the cerebral blood flow correlation amount time series of the estimation target 901 in the estimation cycles is started is referred to as the estimation start time. Hereinafter, a period from the acquisition of the cerebral blood flow correlation amount time series to the next acquisition of the cerebral blood flow correlation amount time series is referred to as a unit period.

The loss-of-consciousness estimation system 100 executes each of later-described out-of-range data determination processing and ventricular state estimation processing once each time the cerebral blood flow correlation amount time series is acquired after the estimation start time.

After the probability acquisition start time, the loss-of-consciousness estimation system 100 further executes each of later-described loss-of-consciousness probability acquisition processing and loss-of-consciousness determination processing once in addition to out-of-range data determination processing and ventricular state estimation processing every time the cerebral blood flow correlation amount time series is acquired.

The probability acquisition start time is the time when the condition of the ventricle of the estimation target 901 is determined as being abnormal for the first time since the loss-of-consciousness estimation system 100 started the acquisition of the brain correlation amount time series of the estimation target 901.

Also, the loss-of-consciousness estimation system 100 executes later-described measurement reliability estimation processing after the probability acquisition start time at the latest.

Hereinafter, with reference to FIG. 1, the out-of-range data determination processing, the ventricular state estimation processing, the measurement reliability estimation processing, the loss-of-consciousness probability acquisition processing, and the loss-of-consciousness determination processing will be described together with the description of the overview of the loss-of-consciousness estimation system 100.

The loss-of-consciousness estimation system 100 repeatedly executes the out-of-range data determination processing in an estimation cycle after the estimation start time. The out-of-range data determination processing is processing for determining whether or not the cerebral blood flow correlation amount indicated by each piece of data of the cerebral blood flow correlation amount time series is out of the range corresponding to the time position of each piece of data (hereinafter referred to as “threshold region”) based on the cerebral blood flow correlation amount time series. The time position is a position in the time axis direction of each piece of data of the cerebral blood flow correlation amount time series (hereinafter referred to as “cerebral blood flow correlation amount point data”).

The loss-of-consciousness estimation system 100 determines whether or not each piece of cerebral blood flow correlation amount point data is out-of-range data by executing the out-of-range data determination processing. The out-of-range data is cerebral blood flow correlation amount point data that is out of range of the threshold region.

The threshold region is a range having at least an upper limit value and a lower limit value. The upper limit value of the threshold region is hereinafter referred to as an upper threshold value. The lower limit value of the threshold region is hereinafter referred to as a lower threshold value.

The threshold region is determined according to the distribution of the cerebral blood flow correlation amount point data within a period of a first length including the time position where the threshold region is determined. Hereinafter, the period of the first length is referred to as a first period. The first length (i.e., the length of the first period) need only be an amount of time having a predetermined number of more of pieces of cerebral blood flow correlation quantity point data within the first period. The first length is, for example, 3 seconds.

The upper threshold value is, for example, (M+V), where the average value of the cerebral blood flow correlation amount indicated by the cerebral blood flow correlation amount point data in the first period including the time position where the threshold region is determined is M, and the standard deviation is V. The lower threshold value is, for example, (M−V), where the average value of the cerebral blood flow correlation amount indicated by the cerebral blood flow correlation amount point data in the first period including the time position where the threshold region is determined is M and the standard deviation is V.

Note that the calculation of the upper limit value of the threshold region and the lower limit value of the threshold region is not necessarily limited to the average value M and the standard deviation V, and the detection sensitivity may be adjusted by multiplying the standard deviation V by a constant (correction value), and may be converted using a function. Also, the upper limit value of the threshold region and the lower limit value of the threshold region may be calculated based on the variance (of the cerebral blood flow correlation amount), and may be adjusted using a device or environmental data that is not a biological signal, and the continuity (whether or not there is a flaw in the observed values).

Being out of range of the threshold region means that a value is less than the lower threshold value or greater than the upper threshold value.

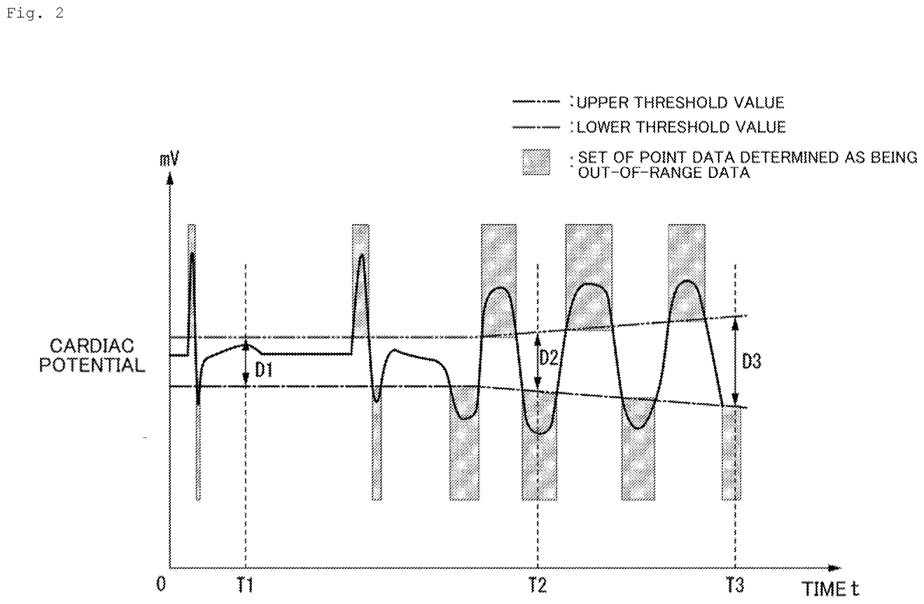

FIG. 2 is a diagram showing an upper threshold value, a lower threshold value, a threshold region, and out-of-range data in the first embodiment. FIG. 2 shows a cardiac potential time series as an example of the cerebral blood flow correlation amount time series. The horizontal axis in FIG. 2 shows the elapsed time from the time of the origin. The vertical axis in FIG. 2 shows the cardiac potential. FIG. 2 shows the upper threshold value and the lower threshold value. As shown in FIG. 2, the upper threshold value and the lower threshold value are not necessarily the same at all times.

In FIG. 2, the ranges of the cardiac potential indicated by D1, D2, and D3 are the threshold regions at time T1, time T2, and time T3, respectively. As shown in FIG. 2, the range of the cardiac potential indicated by the threshold region is not necessarily the same at all times. FIG. 2 shows a set of pieces of cerebral blood flow correlation amount point data determined as being out-of-range data.

The description of FIG. 1 is returned to. The loss-of-consciousness estimation system 100 executes the ventricular state estimation processing in an estimation cycle after the estimation start time. The ventricular state estimation processing is executed after the out-of-range data determination processing is executed in the unit period. The ventricular state estimation processing includes normal ventricular state estimation processing and abnormal ventricular state estimation processing. In the ventricular state estimation processing, the normal ventricular state estimation processing is first executed, and then the abnormal ventricular state estimation processing is executed according to the result of the normal ventricular state estimation processing.

In the normal ventricular state estimation processing, first, it is determined whether or not the ventricular state of the estimation target 901 is normal based on the determination result of the out-of-range data determination processing. In the normal ventricular state estimation processing, if it is determined that the ventricular state of the estimation target 901 is normal, it is estimated that the ventricular state of the estimation target 901 is normal.

The abnormal ventricular state estimation processing is executed when the state of the ventricle of the estimation target 901 is estimated as not being normal (that is, abnormal) through the normal ventricular state estimation processing. The abnormal ventricular state estimation processing is processing for estimating which of predetermined abnormal ventricular states the ventricular state of the estimation target 901 is, based on the cerebral blood flow correlation amount time series.

An abnormal ventricular state is a ventricular state associated with loss of consciousness. The predetermined abnormal ventricular state may be any ventricular state, as long as it is a ventricular state associated with loss of consciousness. An abnormal ventricular state is, for example, a ventricular state in which ventricular tachycardia occurs. The abnormal ventricular state may be, for example, a ventricular state in which ventricular fibrillation occurs. Hereinafter, for the sake of simplicity of description, the loss-of-consciousness estimation system 100 will be described taking, as an example, a case in which the predetermined abnormal ventricular state is ventricular tachycardia and a case in which the predetermined abnormal ventricular state is ventricular fibrillation.

In this manner, the loss-of-consciousness estimation system 100 estimates the ventricular state of the estimation target 901 by executing the ventricular state estimation processing.

If the ventricular state is estimated as not being normal by executing the ventricular state estimation processing for the first time after the estimation start time, the loss-of-consciousness estimation system 100 transmits a warning to a transmission destination. Hereinafter, the warning transmitted to the transmission destination when the ventricular state is determined as being abnormal through the ventricular state estimation processing is referred to as a first warning. Specifically, the first warning is information indicating that there is a high likelihood that the estimation target 901 has lost consciousness.

The transmission destination is, for example, a manager 902 or the estimation target 901. The transmission destination is not only the manager 902 or the estimation target 901, but may also be a safety apparatus such as an autopilot apparatus or an autopilot system.

After it is first estimated that the ventricular state is not normal through the normal ventricular state estimation processing after the start of estimation, the loss-of-consciousness estimation system 100 starts repeatedly executing the loss-of-consciousness probability acquisition processing in the estimation cycle. For this reason, the time when it is first estimated that the ventricular state is not normal through by the normal ventricular state estimation processing after the estimation start time is the probability acquisition start time. The loss-of-consciousness probability acquisition processing is processing for acquiring the loss-of-consciousness probability.

The loss-of-consciousness probability is the probability that the estimation target 901 has already lost consciousness, and is a probability corresponding to the elapsed time from the start of probability acquisition, the change in the measurement environment, and change in the state of the estimation target 901 that appears as a change in the cerebral blood flow correlation time series acquired by the loss-of-consciousness estimation system 100. The loss-of-consciousness probability is an indicator of the likelihood of loss-of-consciousness.

Hereinafter, the state of the estimation target 901 that appears as a change in the cerebral blood flow correlation time series acquired by the loss-of-consciousness estimation system 100 is referred to as a loss-of-consciousness-related state. Specifically, it is estimated through the ventricular state estimation processing whether or not a change in the loss-of-consciousness-related state has occurred.

Specifically, the measurement environment is the state of an apparatus (hereinafter referred to as “acquisition-related apparatus”) related to the acquisition of the cerebral blood flow correlation amount time series of the estimation target 901, such as the biological sensor 1. The acquisition-related apparatus includes, for example, a communication path if the information of the biological sensor 1 is to be transmitted to the transmission destination using the communication path in the loss-of-consciousness estimation system 100.

A change in the measurement environment is a change in the state of the acquisition-related apparatus. The change in the measurement environment is, for example, a change in the operation of the acquisition-related apparatus from a normal operation to an abnormal operation. Abnormal operation occurs when, for example, the acquisition-related apparatus is broken. The change in the acquisition environment may be, for example, a change in which the electrode of the biological sensor 1 comes off of the estimation target 901 when the biological sensor 1 is an apparatus that measures the cerebral blood flow correlation amount using the electrode attached to the estimation target 901.

If the operation of the acquisition-related apparatus is abnormal, the reliability (hereinafter referred to as “measurement reliability”) of the cerebral blood flow correlation amount time series output by the acquisition-related apparatus is lower than that in the case where the operation of the acquisition-related apparatus is normal. The loss-of-consciousness probability is the probability that the estimation target 901 has already lost consciousness, and therefore if there is no change in the loss-of-consciousness-related state of the estimation target 901 but the operation of the acquisition-related apparatus changes from normal to abnormal, the loss-of-consciousness probability decreases.

Also, the loss-of-consciousness probability is the probability that the estimation target 901 has already lost consciousness, and therefore if there is no change in the measurement environment but the loss-of-consciousness-related state changes from an abnormal state to a normal state, the loss-of-consciousness probability decreases. The state in which the loss-of-consciousness-related state is abnormal is, for example, a state in which the movement of the heart of the estimation target 901 is abnormal.

Incidentally, if there is no change in the measurement environment and the state of the estimation target 901 after the start of probability acquisition, the probability that the estimation target 901 will lose consciousness increases with the passage of time. For this reason, if there is no change in the measurement environment and the loss-of-consciousness-related state of the estimated target 901, the loss-of-consciousness probability increases with the passage of time.

The loss-of-consciousness probability is also a value indicating the reliability of the estimation result of the ventricular state estimation processing. For example, if there is no change in the measurement environment and the ventricular state of the estimation target 901 after the start of probability acquisition, the displayed loss-of-consciousness probability indicates a probability that increases with the passage of time.

One specific example of the display of the loss-of-consciousness probability is graph G1 in FIG. 1. The horizontal axis of graph G1 shows time. The vertical axis of graph G1 shows the loss-of-consciousness probability. The time t1 in the graph G1 is the probability acquisition start time. For this reason, the time t1 in the graph G1 is an example of the time when the loss-of-consciousness probability acquisition processing is started. The origin on the time axis of graph G1 is an example of the estimation start time.

Note that the loss-of-consciousness probability is merely a probability. For this reason, even if the loss-of-consciousness probability is high, the estimation target 901 may not have lost consciousness.

After the probability acquisition start time, the loss-of-consciousness estimation system 100 repeatedly executes the loss-of-consciousness determination processing in the estimation cycles. The loss-of-consciousness determination processing is processing for determining whether or not the loss-of-consciousness probability is a predetermined probability (hereinafter referred to as “reference probability”) or more.

If the loss-of-consciousness probability is the reference probability or more, the loss-of-consciousness estimation system 100 transmits a warning to the transmission destination. Hereinafter, the warning transmitted to the transmission destination when the loss-of-consciousness probability is the reference probability or more will be referred to as a second warning. Specifically, the second warning is information indicating that the probability that the estimation target 901 has lost consciousness is high.

If the loss-of-consciousness probability is the reference probability or more, the loss-of-consciousness estimation system 100 may transmit not only the second warning but also the loss-of-consciousness probability itself to the transmission destination.

The transmission destination is, for example, the manager 902 or the estimation target 901. The transmission destination may be not only the manager 902 or the estimation target 901, but also a safety apparatus such as an autopilot apparatus or an autopilot system.

If the loss-of-consciousness probability is less than the reference probability, the loss-of-consciousness estimation system 100 executes the loss-of-consciousness probability acquisition processing without transmitting a warning to the transmission destination. However, after the loss-of-consciousness probability has reached the reference probability or more even once, the loss-of-consciousness estimation system 100 may transmit the second warning even if the loss-of-consciousness probability is less than the reference probability.

The loss-of-consciousness estimation system 100 executes the measurement reliability estimation processing after the probability acquisition start time, at the latest. The measurement reliability estimation processing is processing for estimating the measurement reliability based on the cerebral blood flow correlation amount time series. The measurement reliability estimation processing is processing for estimating that the measurement reliability is lower than in the case where the acquisition-related apparatus has not broken down if, for example, the value of a predetermined index indicated by the cerebral blood flow correlation amount time series is a value at the time of breakdown of the acquisition-related apparatus.

The predetermined index indicated by the cerebral blood flow correlation amount time series is, for example, the cardiac potential indicated by the cardiac potential time series. For example, if the cardiac potential is a value greater than or equal to the measurement limit of the apparatus, it is estimated that the acquisition-related apparatus has broken down in the measurement reliability estimation processing.

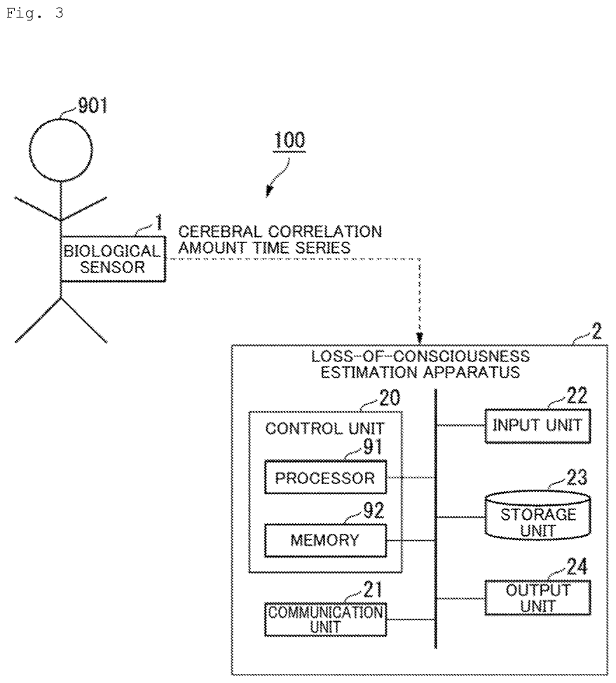

FIG. 3 is a diagram showing an example of the system configuration of the loss-of-consciousness estimation system 100 of the first embodiment. The loss-of-consciousness estimation system 100 includes the biological sensor 1 and the loss-of-consciousness estimation apparatus 2.

The biological sensor 1 repeatedly measures the cerebral blood flow correlation amount to be measured at predetermined time intervals that are shorter than the estimation cycle. The measurement result of the biological sensor 1 is the cerebral blood flow correlation amount time series. The biological sensor 1 outputs the measurement result to the loss-of-consciousness estimation apparatus 2. The biological sensor 1 is, for example, a heartbeat sensor.

The loss-of-consciousness estimation apparatus 2 repeatedly acquires the cerebral blood flow correlation amount time series acquired by the biological sensor 1 in the estimation cycle. The loss-of-consciousness estimation apparatus 2 executes the out-of-range data determination processing, the ventricular state estimation processing, the loss-of-consciousness probability acquisition processing, and the loss-of-consciousness determination processing based on the cerebral blood flow correlation amount time series.

The loss-of-consciousness estimation apparatus 2 includes a control unit 20 including a processor 91 such as a CPU (Central Processing Unit) and a memory 92, which are connected by a bus, and executes a program. The loss-of-consciousness estimation apparatus 2 functions as an apparatus including the control unit 20, a communication unit 21, an input unit 22, a storage unit 23, and an output unit 24 by executing the program.

More specifically, in the loss-of-consciousness estimation apparatus 2, the processor 91 reads out a program stored in the storage unit 23, and stores the read-out program in the memory 92. Due to the processor 91 executing the program stored in the memory 92, the loss-of-consciousness estimation apparatus 2 functions as an apparatus including the control unit 20, the communication unit 21, the input unit 22, the storage unit 23, and the output unit 24.

The control unit 20 controls the operation of each functional unit included in the loss-of-consciousness estimation apparatus 2. The control unit 20 acquires, for example, the cerebral blood flow correlation amount time series in the estimation cycle. The reciprocal of the estimation cycle (i.e., the sampling rate) is, for example, 1 kHz. The control unit 20 executes, for example, the out-of-range data determination processing, the ventricular state estimation processing, the loss-of-consciousness probability acquisition processing, and the loss-of-consciousness determination processing.

The communication unit 21 is constituted by including a communication interface for connecting the loss-of-consciousness estimation apparatus 2 to the biological sensor 1. The communication unit 21 communicates with the biological sensor 1 via, for example, a network. The communication unit 21 acquires the cerebral blood flow correlation amount time series from the biological sensor 1 by communicating with the biological sensor 1.

The input unit 22 is constituted by including an input apparatus such as a mouse, a keyboard, and a touch panel. The input unit 22 may also be configured as an interface for connecting these input apparatuses to the loss-of-consciousness estimation apparatus 2.

The storage unit 23 is formed using a storage apparatus such as a magnetic hard disk apparatus or a semiconductor storage apparatus. The storage unit 23 stores various types of information related to the loss-of-consciousness estimation apparatus 2. The storage unit 23 stores, for example, the history of the cerebral blood flow correlation amount time series output by the biological sensor 1. The storage unit 23 stores, for example, a program for controlling the operation of the loss-of-consciousness estimation apparatus 2 in advance.

The storage unit 23 stores, for example, information indicating the estimation start time. The storage unit 23 stores, for example, information indicating the probability acquisition start time. The storage unit 23 stores, for example, the history of the loss-of-consciousness probability.

The output unit 24 is constituted by including a display apparatus such as a CRT (Cathode Ray Tube) display, a liquid crystal display, and an organic EL (Electro-Luminescence) display, and an information output apparatus such as an audio output apparatus such as a speaker. The output unit 24 may also be configured as an interface for connecting these output apparatuses to the loss-of-consciousness estimation apparatus 2. The output unit 24 outputs information regarding the loss-of-consciousness estimation apparatus 2. The output unit 24 outputs, for example, the information input to the input unit 22. The output unit 24 outputs, for example, the first warning. The output unit 24 outputs, for example, the second warning. The output unit 24 outputs, for example, the loss-of-consciousness probability. The output unit 24 outputs, for example, the measurement reliability. The information output by the output unit 24 is acquired by the transmission destination such as the manager 902 or the estimation target 901.

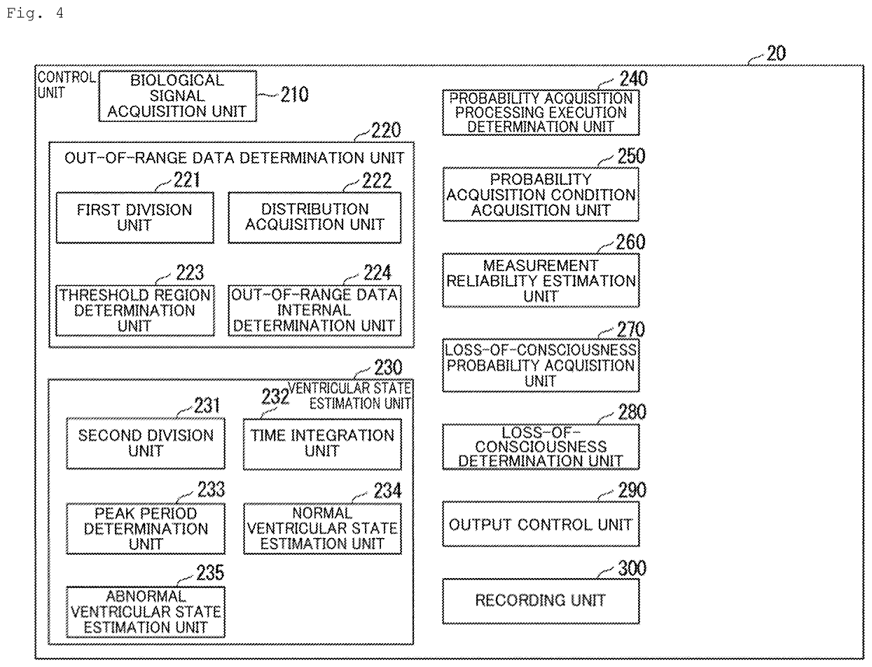

FIG. 4 is a diagram showing an example of a functional configuration of the control unit 20 in the first embodiment. The control unit 20 includes a time series acquisition unit 210, an out-of-range data determination unit 220, a ventricular state estimation unit 230, a probability acquisition processing execution determination unit 240, a probability acquisition condition acquisition unit 250, a measurement reliability estimation unit 260, and a loss-of-consciousness probability acquisition unit 270, a loss-of-consciousness determination unit 280, an output control unit 290, and a recording unit 300.

The time series acquisition unit 210 repeatedly acquires the cerebral blood flow correlation amount time series in the estimation cycle via the communication unit 21.

The out-of-range data determination unit 220 executes the out-of-range data determination processing on the cerebral blood flow correlation amount time series acquired by the time series acquisition unit 210. The out-of-range data determination unit 220 executes the threshold region determination processing as part of the out-of-range data determination processing. The threshold region determination processing is processing for determining the threshold region of each time position. More specifically, the threshold region determination processing is processing for determining the threshold region to be determined according to the cerebral blood flow correlation amount point data distribution in the first period including the time position where the threshold region is determined.

The out-of-range data determination unit 220 includes a first division unit 221, a distribution acquisition unit 222, a threshold region determination unit 223, and an out-of-range data internal determination unit 224.

The first division unit 221 divides the cerebral blood flow correlation amount time series in the time axis direction by a plurality of first periods.

The distribution acquisition unit 222 acquires the cerebral blood flow correlation amount point data statistical amount for each first period. The cerebral blood flow correlation amount point data statistical amount is the amount for each first period, and is a statistical amount relating to the distribution of the cerebral blood flow correlation amount indicated by the cerebral blood flow correlation amount point data belonging to each first period. The statistical amount related to the distribution may be any statistical amount as long as it can represent the distribution.

Statistical amounts related to the distribution are, for example, the mean and the deviation. The deviation may be any statistical amount as long as it is a statistical amount indicating a deviation from the average value, and may be, for example, a standard deviation. Hereinafter, for the sake of simplicity of description, the loss-of-consciousness estimation system 100 will be described taking, as an example, a case where the cerebral blood flow correlation amount point data statistical amount is the average value and the standard deviation.

The threshold region determination unit 223 determines the threshold region for each first period based on the cerebral blood flow correlation amount point data statistical amount.

The threshold region determination processing is processing in which the threshold region is determined in each first period by a series of processing executed by the first division unit 221, the distribution acquisition unit 222, and the threshold region determination unit 223.

The out-of-range data internal determination unit 224 determines whether or not each piece of cerebral blood flow correlation amount point data is out-of-range data by using the threshold region determined through the threshold region determination processing.

The out-of-range data determination processing is processing for determining whether or not each piece of cerebral blood flow correlation amount point data is out-of-range data through the threshold region determination processing and the processing executed by the out-of-range data internal determination unit 224.

The ventricular state estimation unit 230 executes the ventricular state estimation processing. The ventricular state estimation unit 230 includes a second division unit 231, a time integration unit 232, a peak period determination unit 233, a normal ventricular state estimation unit 234, and an abnormal ventricular state estimation unit 235.

The second division unit 231 divides the cerebral blood flow correlation amount time series by a plurality of second periods. The length of the second period is shorter than the length of the first period, which shares some or all of the time positions. The length of the second period is, for example, 200 milliseconds.

The time integration unit 232 integrates the length of the period including only the out-of-range data for each second period. Hereinafter, the total value (i.e., the integrated time) of the lengths of the periods including only the out-of-range data is referred to as the out-of-range time.

The peak period determination unit 233 determines whether or not the out-of-range time exceeds a predetermined time (hereinafter referred to as “threshold time”) for each second period. The threshold time is a time that is shorter than the length of the second period. If the length of the second period is 200 milliseconds, the threshold time is, for example, 50 milliseconds. Hereinafter, the second period in which the out-of-range time is determined as being longer than the threshold time by the peak period determination unit 233 will be referred to as a peak period.

The normal ventricular state estimation unit 234 executes the normal ventricular state estimation processing. Specifically, the normal ventricular state estimation unit 234 determines whether the ventricular state of the estimation target 901 is normal or abnormal based on the appearance of the peak period in the cerebral blood flow correlation amount time series.

Specifically, the normal ventricular state estimation unit 234 determines that the ventricular state of the estimation target 901 is not normal if, for example, a predetermined number of second periods that are adjacent in the time axis direction are peak periods. That is, the normal ventricular state estimation unit 234 determines that the ventricular state of the estimation target 901 is not normal if the condition that a predetermined number of peak periods are continuous in the time axis direction is satisfied.

The predetermined number is, for example, five instances. For example, if the length of the second period is 200 milliseconds and the threshold time is 50 milliseconds, and if five second periods that are adjacent in the time axis direction are peak periods, the normal ventricular state estimation unit 234 determines that the ventricular state of the estimation target 901 is not normal.

Note that medically, the ventricular state of the estimation target 901 is a state in which ventricular tachycardia or ventricular fibrillation has occurred if the length of the second period is 200 ms and the threshold time is 50 ms, and if five second periods that are adjacent in the time axis direction are peak periods. That is, the condition that five second peak periods that are adjacent in the time axis direction are peak periods when the length of the second period is 200 milliseconds and the threshold time is 50 milliseconds is a condition under which the ventricular state of the estimation target 901 is not normal, from a medical point of view.

Incidentally, the state in which the ventricular state of the estimation target 901 is normal is equivalent to a condition under which the heartbeat interval is within a medically-known predetermined range (hereinafter referred to as “reference frequency range”), from a medical point of view.

For this reason, the processing for determining whether the ventricular state of the estimation target 901 by the normal ventricular state estimation unit 234 is normal or abnormal is equivalent to processing for determining whether or not the heartbeat interval is within the reference frequency range.

If it is determined that the ventricular state of the estimation target 901 is normal, the normal ventricular state estimation unit 234 estimates that the ventricular state of the estimation target 901 is normal.

The abnormal ventricular state estimation unit 235 executes the abnormal ventricular state estimation processing if it has been determined by the normal ventricular state estimation unit 234 that the ventricular state of the estimation target 901 is abnormal.

Specifically, in the abnormal ventricular state estimation processing, the abnormal ventricular state estimation unit 235 first acquires a value (hereinafter referred to as “heartbeat interval-related value”) related to the heartbeat interval indicated by the cerebral blood flow correlation amount time series based on the cerebral blood flow correlation amount time series. In the abnormal ventricular state estimation processing, the abnormal ventricular state estimation unit 235 then estimates the abnormal ventricular state of the estimation target 901 based on the acquired heartbeat interval-related value.

The heartbeat interval-related value is, for example, the heartbeat interval itself. If the heartbeat interval-related value is the heartbeat interval, the abnormal ventricular state estimation unit 235 estimates that the ventricular state of the estimation target 901 is a state of ventricular fibrillation if the frequency of appearance of the peak period is higher than the first reference frequency. The first reference frequency is the highest value within the reference frequency range. Also, if the heartbeat interval-related value is the heartbeat interval, the abnormal ventricular state estimation unit 235 estimates that the ventricular state of the estimation target 901 is a state of ventricular tachycardia if the frequency of appearance of the peak period is less than the second reference frequency. The second reference frequency is the minimum value within the reference frequency range.

The heartbeat interval-related value may also be, for example, cardiac output. The heartbeat interval-related value may also be, for example, the organ blood flow rate.

Note that the heartbeat interval is acquired through, for example, a method of acquiring an R spine peak point acquired from a combination of a threshold value and an inflection point and measuring a time interval between the R spine peak point and the next R spine peak point.

Note that the cardiac output per hour is estimated based on, for example, the standard value of human stroke volume observed using a cardiac output meter and a value obtained by multiplying the stroke volume corrected using Frank-Starling's law and the heart rate.

Note that the organ blood flow rate is estimated using information (hereinafter referred to as “organ blood flow rate-related information”) stored in advance in the storage unit 23, the information indicating the relationship between blood pressure observed using a deep blood flow meter, a continuous sphygmomanometer, and a posture or environmental sensor, and the standard blood flow rate of the organ. More specifically, the organ blood flow rate is estimated by estimating the ratio between the cerebral blood flow and the blood flow of other organs, based on the organ blood flow rate-related information. The organ blood flow rate is an amount related to the flow rate and distribution of blood flowing in an organ closely related to loss of consciousness, such as the brain and heart. Being closely related to loss of consciousness such as the brain and heart refers to organs involved in blood circulation of cranial nerve tissue and circulatory regulation function, and specifically means the blood vessels in the brain, the heart, the head and neck, and the thorax, the lungs, and the circulatory regulation function thereof.

The series of processing performed by each functional unit included in the ventricular state estimation unit 230 from the second division unit 231 to the abnormal ventricular state estimation unit 235 is an example of the ventricular state estimation processing.

The probability acquisition processing execution determination unit 240 determines whether or not to execute the loss-of-consciousness probability acquisition processing. Specifically, the probability acquisition processing execution determination unit 240 determines that the loss-of-consciousness probability acquisition processing is to be executed if the timing of determining whether or not to execute the loss-of-consciousness probability acquisition processing is after the probability acquisition start time. The probability acquisition processing execution determination unit 240 determines that the loss-of-consciousness probability acquisition processing is not to be executed if the timing of determining whether or not to execute the loss-of-consciousness probability acquisition processing is not after the probability acquisition start time.

The probability acquisition condition acquisition unit 250 acquires information indicating the probability determination condition based on the estimation result of the ventricular state estimation unit 230 and the information indicating the relationship between the ventricular state and the probability determination condition stored in the storage unit 23 in advance. The probability determination condition is a condition for each combination of the elapsed time from the start of probability acquisition, the change in the measurement environment, and the change in the state of the estimation target 901 that appears as a change in the cerebral blood flow correlation time series, and includes conditions that provide the amount of change in the loss-of-consciousness probability.

The probability determination condition is, for example, a condition that provides the amount of change in the loss-of-consciousness probability when the elapsed time from the probability acquisition start time has changed by the unit time. The probability determination condition also includes a condition indicating an initial value of the loss-of-consciousness probability. The initial value of the loss-of-consciousness probability is the loss-of-consciousness probability immediately after the probability acquisition start time.

The measurement reliability estimation unit 260 executes the measurement reliability estimation processing. The measurement reliability estimation unit 260 estimates the measurement reliability by executing the measurement reliability estimation processing.

The loss-of-consciousness probability acquisition unit 270 executes the loss-of-consciousness probability acquisition processing. Specifically, the loss-of-consciousness probability acquisition unit 270 acquires the loss-of-consciousness probability based on the probability acquisition condition acquired by the probability acquisition condition acquisition unit 250, the measurement reliability estimated by the measurement reliability estimation unit 260, the elapsed time from the probability acquisition start time, and the estimation result of the ventricular state estimation unit 230 after the probability acquisition start time.

For example, if the measurement reliability is the reference reliability or more and if the ventricular state estimation unit 230 determines that the ventricular state of the estimation target 901 is abnormal, the loss-of-consciousness probability acquired by the loss-of-consciousness probability acquisition unit 270 is higher than the loss-of-consciousness probability acquired in the immediately-previous unit period. The reference reliability is a predetermined value.

For example, if the measurement reliability is the reference reliability or more and if the ventricular state estimation unit 230 determines that the ventricular state of the estimation target 901 is normal, the loss-of-consciousness probability acquired by the loss-of-consciousness probability acquisition unit 270 is lower than the loss-of-consciousness probability acquired in the immediately-previous unit period.

For example, if the measurement reliability is less than the reference reliability, the loss-of-consciousness probability acquired by the loss-of-consciousness probability acquisition unit 270 is lower than the loss-of-consciousness probability acquired in the immediately-previous unit period.

The loss-of-consciousness determination unit 280 executes the loss-of-consciousness determination processing.

The output control unit 290 controls the operation of the output unit 24. For example, the output control unit 290 controls the operation of the output unit 24 to cause the output unit 24 to output the first warning. For example, the output control unit 290 controls the operation of the output unit 24 to cause the output unit 24 to output the second warning. For example, the output control unit 290 controls the operation of the output unit 24 to cause the output unit 24 to output the loss-of-consciousness probability. For example, the output control unit 290 controls the operation of the output unit 24 to cause the output unit 24 to output the measurement reliability.

The recording unit 300 records various types of information in the storage unit 23. The recording unit 300 records, for example, information indicating the estimation start time in the storage unit 23. The recording unit 300 records, for example, information indicating the probability acquisition start time in the storage unit 23. The recording unit 300 records, for example, the estimation result of the ventricular state estimation unit 230 in the storage unit 23. The recording unit 300 records, for example, the probability condition acquired by the probability acquisition condition acquisition unit 250 in the storage unit 23.

The recording unit 300 records, for example, the measurement reliability estimated by the measurement reliability estimation unit 260 in the storage unit 23. The recording unit 300 records, for example, the loss-of-consciousness probability acquired by the loss-of-consciousness probability acquisition unit 270 in the storage unit 23. The recording unit 300 records, for example, the determination result of the loss-of-consciousness determination unit 280 in the storage unit 23.

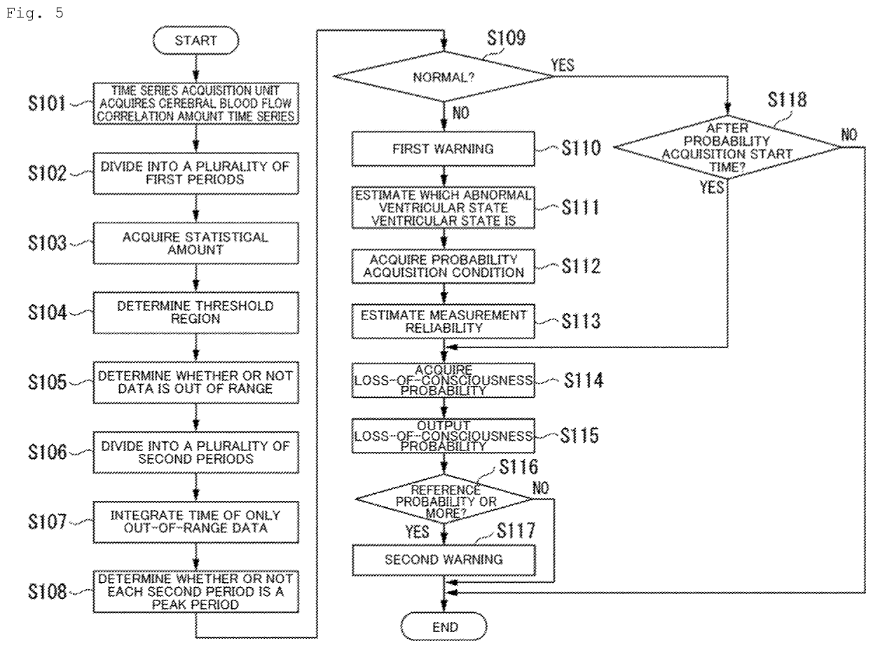

FIG. 5 is a flowchart showing an example of a flow of processing executed by the loss-of-consciousness estimation system 100 in the first embodiment. FIG. 5 is a flowchart showing a flow of processing executed in one unit period. For this reason, in the loss-of-consciousness estimation system 100, the flowchart shown in FIG. 5 is repeatedly executed in the estimation cycle.

Also, during the execution of the flowchart shown in FIG. 5 by the loss-of-consciousness estimation system 100, the processing for acquiring the cerebral blood flow correlation amount time series by the biological sensor 1 is repeatedly executed at a predetermined timing.

Also, during the execution of the flowchart shown in FIG. 5 by the loss-of-consciousness estimation system 100, the processing for acquiring the cerebral blood flow correlation amount time series by the time series acquisition unit 210 is also repeatedly executed at predetermined time intervals. Also, after the execution of each process of the flowchart shown in FIG. 5, the recording unit 300 records information indicating the execution result of each process in the storage unit 23. For example, the recording unit 300 records information indicating the probability acquisition start time in the storage unit 23 at the probability acquisition start time.

The time series acquisition unit 210 acquires the cerebral blood flow correlation amount time series acquired by the biological sensor 1 (step S101). The processing for the unit period is started through the execution of the processing of step S101.

Following step S101, the first division unit 221 divides the cerebral blood flow correlation amount time series in the time axis direction by a plurality of first periods (step S102). Next, the distribution acquisition unit 222 acquires the cerebral blood flow correlation amount point data statistical amount for each first period (step S103). Next, the threshold region determination unit 223 determines the threshold region for each first period based on the cerebral blood flow correlation amount point data statistical amount (step S104).

Next, the out-of-range data internal determination unit 224 determines whether or not each piece of cerebral blood flow correlation amount point data in the cerebral blood flow correlation amount time series is out-of-range data (step S105). The processing of steps S102 to S104 is the threshold region determination processing, and the processing of steps S102 to S105 is the out-of-range data determination processing. As shown in the flow of steps S102 to S105, the threshold region determination processing is executed before the execution of step S105.

Next, the second division unit 231 divides the cerebral blood flow correlation amount time series by a plurality of second periods (step S106). Next, the time integration unit 232 acquires the out-of-range time for each second period (step S107). Next, the peak period determination unit 233 determines whether or not each second period is a peak period (step S108).

Next, the normal ventricular state estimation unit 234 determines whether the ventricular state of the estimation target 901 is normal or abnormal (step S109). If the ventricular state of the estimation target 901 is not normal (step S109: NO), the output control unit 290 controls the operation of the output unit 24 to cause the output unit 24 to output the first warning (step S110). Next, the abnormal ventricular state estimation unit 235 executes the abnormal ventricular state estimation processing (step S111). The abnormal ventricular state estimation unit 235 estimates which abnormal ventricular state is the ventricular state of the estimation target 901 by executing the abnormal ventricular state estimation processing.

The series of processes from step S106 to step S111 is an example of the ventricular state estimation processing.

Following step S111, the probability acquisition condition acquisition unit 250 acquires the probability acquisition condition based on the estimation result of the abnormal ventricular state estimation unit 235 (step S112). Next, the measurement reliability estimation unit 260 estimates the measurement reliability based on the cerebral blood flow correlation amount time series (step S113). Next, the loss-of-consciousness probability acquisition unit 270 acquires the loss-of-consciousness probability (step S114).

Next, the operation of the output unit 24 is controlled to cause the output unit 24 to output the loss-of-consciousness probability through display or the like (step S115). Next, the loss-of-consciousness determination unit 280 determines whether or not the loss-of-consciousness probability is the reference probability or more (step S116).

If the loss-of-consciousness probability is the reference probability or more (step S116: YES), the output control unit 290 controls the operation of the output unit 24 to cause the output unit 24 to output the second warning (step S117). On the other hand, if the loss-of-consciousness probability is less than the reference probability (step S116: NO), the processing in the unit period ends without the second warning being output.

On the other hand, if the ventricular state of the estimation target 901 is normal (step S109: YES), the probability acquisition processing execution determination unit 240 determines whether or not to execute the loss-of-consciousness probability acquisition processing (step S118). Specifically, the probability acquisition processing execution determination unit 240 determines whether or not the timing of determining whether or not to execute the loss-of-consciousness probability acquisition processing is after the probability acquisition start time.

If the timing of determining whether or not to execute the loss-of-consciousness acquisition probability acquisition processing is after the probability acquisition start time (step S118: YES), the processing of step S114 is executed. On the other hand, if the timing of determining whether or not to execute the loss-of-consciousness probability acquisition processing is not after the probability acquisition start time (step S118: NO), the processing in the unit period ends without the loss-of-consciousness probability being acquired.

Note that if it is determined in step S109 that the ventricular state of the estimation target 901 is normal, the output control unit 290 may also control the operation of the output unit 24 to cause the output unit 24 to output information indicating that the ventricular state of the estimation target 901 is normal.

<Regarding the Relationship Between the Loss-of-Consciousness Probability and the Cerebral Blood Flow Correlation Amount Time Series>

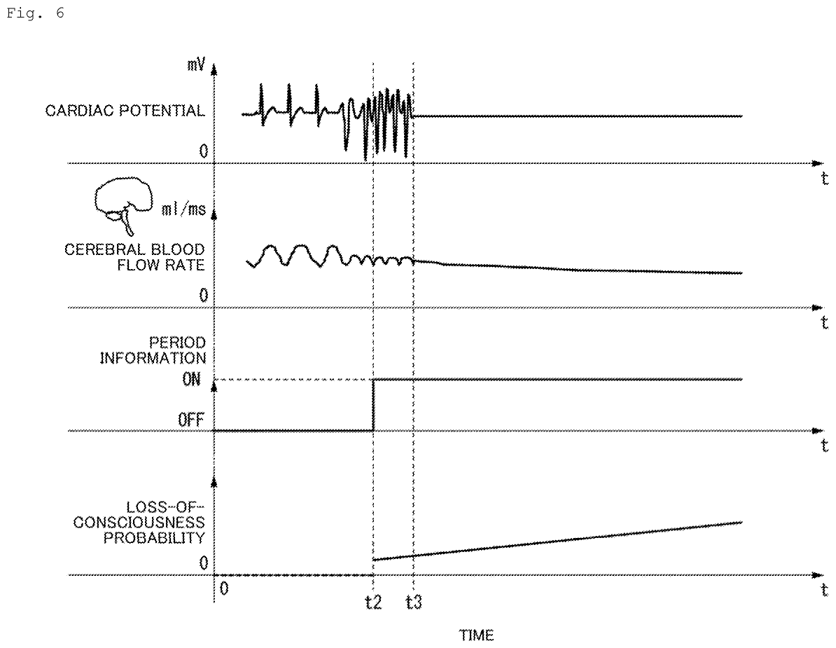

Here, the relationship between the loss-of-consciousness probability and the cerebral blood flow correlation amount time series will be described with reference to FIGS. 6 to 9. Note that the graphs of the loss-of-consciousness probability in FIGS. 6 to 9 are examples of the loss-of-consciousness probability output by the output unit 24 in the processing of step S115. The graphs of the cardiac potential time series and the changes in cerebral blood flow in FIGS. 6 to 9 are examples of cerebral blood flow correlation amount time series.

FIG. 6 is a first explanatory diagram illustrating the relationship between the loss-of-consciousness probability and the cerebral blood flow correlation amount time series in the first embodiment.

FIG. 6 is a diagram in which a cardiac potential time series, a graph of changes in cerebral blood flow, information indicating whether or not it is after the probability acquisition start time (hereinafter referred to as “period information”), and a graph of the loss-of-consciousness probability are shown on the same time axis. The horizontal axis in FIG. 6 indicates the time. For this reason, the horizontal axis in FIG. 6 shows the time elapsed from the time of the origin when the time of the origin is set to 0. The time of the origin in FIG. 6 is an example of the estimation start time. The period information indicates whether or not each time on the horizontal axis is a time after the probability acquisition start time. In FIG. 6, ON indicates that the time is a time after the probability acquisition start time. In FIG. 6, OFF indicates that the time is a time before the probability acquisition start time.

The time t2 in FIG. 6 is an example of the probability acquisition start time. FIG. 6 shows that the cerebral blood flow decreases with time if there is no change over time in the cardiac potential in the period after the probability acquisition start time. FIG. 6 shows that the loss-of-consciousness probability increases in proportion to the time if there is no change over time in the cardiac potential. FIG. 6 shows that cardiac arrest occurred at time t3.

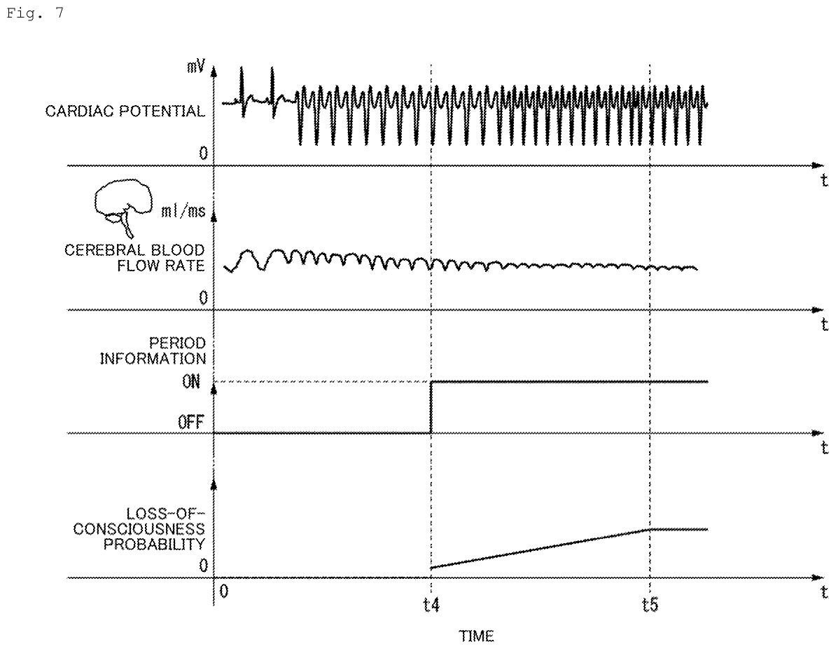

FIG. 7 is a second explanatory diagram illustrating the relationship between the loss-of-consciousness probability and the cerebral blood flow correlation amount time series in the first embodiment.

FIG. 7 is a diagram in which a cardiac potential time series, a graph of changes in cerebral blood flow, period information, and a graph of the loss-of-consciousness probability are shown on the same time axis. The horizontal axis in FIG. 7 indicates the time. For this reason, the horizontal axis in FIG. 7 shows the time elapsed from the time of the origin when the time of the origin is set to 0. The time of the origin in FIG. 7 is an example of the estimation start time. The period information indicates whether or not each time on the horizontal axis is a time after the probability acquisition start time.

The time t4 in FIG. 7 is an example of the probability acquisition start time. FIG. 7 shows an example of a cardiac potential time series in which the heartbeat interval is no longer included in the reference frequency range over time. FIG. 7 shows that the loss-of-consciousness probability increases in proportion to time after the probability acquisition start time up to time t5. Time t5 is the time when the loss-of-consciousness probability reaches the predetermined upper limit of the loss-of-consciousness probability.

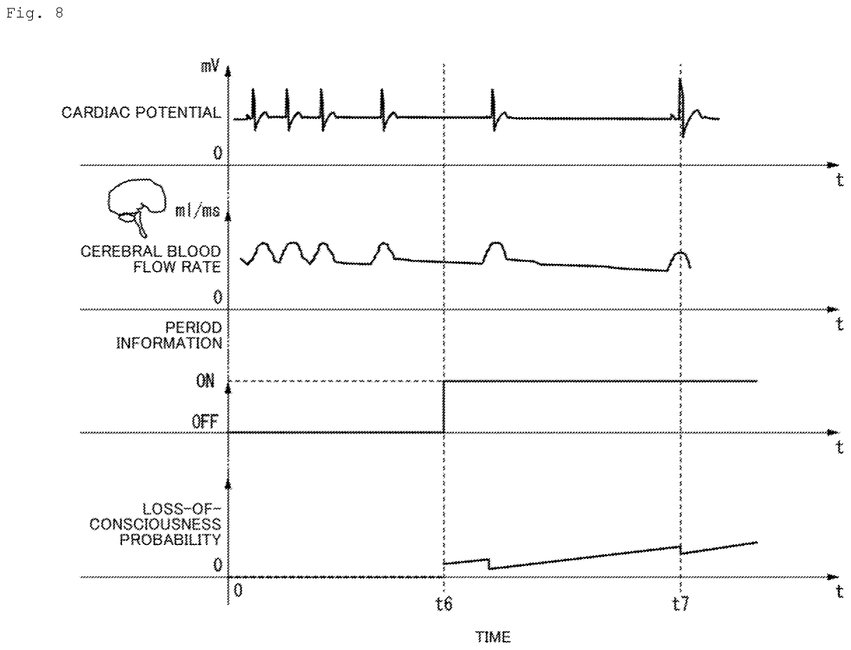

FIG. 8 is a third explanatory diagram illustrating the relationship between the loss-of-consciousness probability and the cerebral blood flow correlation amount time series in the first embodiment.

FIG. 8 is a graph in which a cardiac potential time series, a graph of the change in cerebral blood flow, period information, and a graph of the loss-of-consciousness probability are shown on the same time axis. The horizontal axis in FIG. 8 indicates the time. For this reason, the horizontal axis in FIG. 8 shows the time elapsed from the time of the origin when the time of the origin is set to 0. The time of the origin in FIG. 8 is an example of the estimation start time. The period information indicates whether or not each time on the horizontal axis is a time after the probability acquisition start time.

The time t6 in FIG. 8 is an example of the probability acquisition start time. t7 in FIG. 8 is the time when the ventricular state of the estimation target 901 was determined as being normal by the ventricular state estimation unit due to the occurrence of the heartbeat. For this reason, at t7 in FIG. 8, the loss-of-consciousness probability decreases.

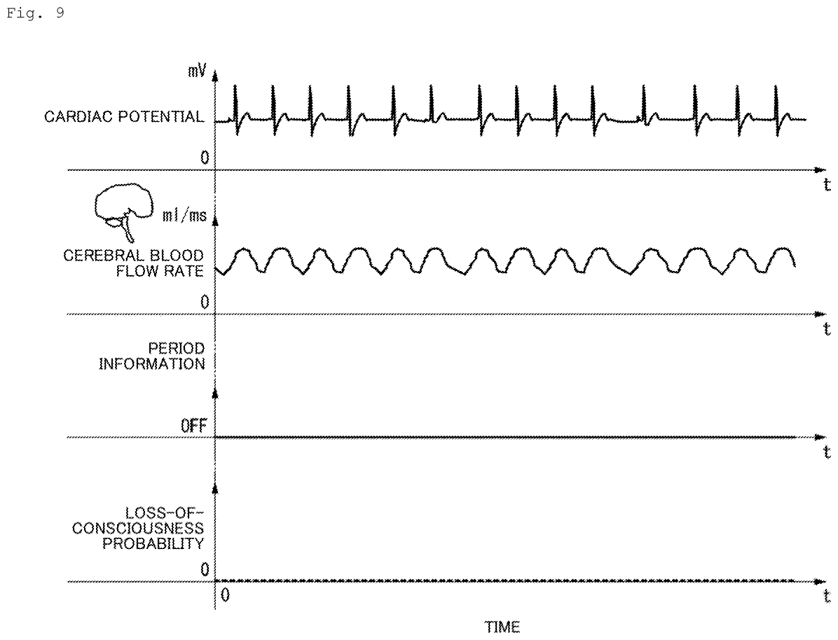

FIG. 9 is a fourth explanatory diagram illustrating the relationship between the loss-of-consciousness probability and the cerebral blood flow correlation amount time series in the first embodiment.

FIG. 9 is a diagram in which a cardiac potential time series, a graph of changes in the cerebral blood flow rate, period information, and a graph of the loss-of-consciousness probability are shown on the same time axis. The horizontal axis in FIG. 9 indicates the time. For this reason, the horizontal axis in FIG. 9 shows the time elapsed from the time of the origin when the time of the origin is set to 0. The time of the origin in FIG. 9 is an example of the estimation start time. The period information indicates whether or not each time on the horizontal axis is a time after the probability acquisition start time.

FIG. 9 shows that since the movement of the heart is stable, the cardiac potential is not disturbed and the cerebral blood flow is also stable. That is, FIG. 9 shows that the ventricular state of the estimation target 901 indicated by the cardiac potential time series continues to be normal. FIG. 9 shows that the period information remains OFF because the ventricular state of the estimation target 901 continues to be normal, and thus the loss-of-consciousness probability is not being acquired.

This is the end of the description of the relationship between the loss-of-consciousness probability and the cerebral blood flow correlation amount time series.

The loss-of-consciousness estimation system 100 of the first embodiment configured in this manner determines the threshold region corresponding to the position of each piece of data in the time axis direction based on the data within a predetermined period including the position of each piece of data in the cerebral blood flow correlation amount time series in the time axis direction. Then, the loss-of-consciousness estimation system 100 estimates the probability that consciousness has already been lost based on the determined threshold region. For this reason, the loss-of-consciousness estimation system 100 can increase the accuracy of estimating the likelihood of loss-of-consciousness of the estimation target 901. For this reason, the loss-of-consciousness estimation system 100 of the first embodiment configured in this manner can reduce the danger posed by loss of consciousness.

Also, the loss-of-consciousness estimation system 100 of the first embodiment configured in this manner outputs the likelihood of the loss of consciousness that is the estimation result using the output unit 24. Since the probability that consciousness has already been lost is output, the estimation target 901 can take actions to reduce the danger posed by loss of consciousness before loss of consciousness occurs. For this reason, the loss-of-consciousness estimation system 100 of the first embodiment configured in this manner can reduce the danger posed by loss of consciousness.

Note that the normal ventricular state estimation unit 234 does not necessarily need to estimate whether or not the ventricular state of the estimation target 901 is normal based on the processing results of the second division unit 231, the time integration unit 232, and the peak period determination unit 233. The normal ventricular state estimation unit 234 may also estimate whether or not the ventricular state of the estimation target 901 is normal using any method as long as it is possible to estimate whether or not the ventricular state of the estimation target 901 is normal. For example, the normal ventricular state estimation unit 234 may also estimate whether or not the ventricular state is normal based on only the frequency of occurrence of extreme values of the cardiac potential.

However, in the case of estimating whether or not the ventricular state is normal based on only the frequency of occurrence of extreme values of the cardiac potential, extreme values caused by some disturbance of the waveform may also be counted in the instance count, resulting in an erroneous estimation result. On the other hand, in the method based on the processing results of the second division unit 231, the time integration unit 232, and the peak period determination unit 233, it is possible to selectively extract continuous waves having a unique vibration that is symmetrical, which is a characteristic of an abnormal signal at the time of ventricular fibrillation originating in the heart. For this reason, the probability of erroneous estimation is lower than that in the case of estimating whether or not the ventricular state is normal based on only the frequency of occurrence of extreme values of the cardiac potential.

For this reason, it is desirable that the ventricular state estimation unit 230 includes a normal ventricular state estimation unit 234 that estimates whether or not the ventricular state of the estimation target 901 is normal based on the processing results of the second division unit 231, the time integration unit 232, and the peak period determination unit 233.

### Second Embodiment

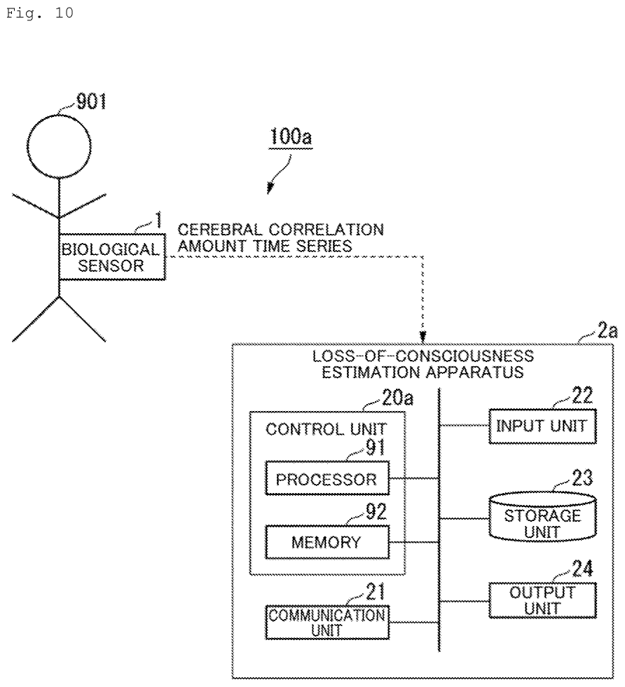

FIG. 10 is a diagram showing an example of a system configuration of a loss-of-consciousness estimation system 100a of a second embodiment. The loss-of-consciousness estimation system 100a differs from the loss-of-consciousness estimation system 100 in that it includes a loss-of-consciousness estimation apparatus 2a instead of the loss-of-consciousness estimation apparatus 2. The loss-of-consciousness estimation apparatus 2a differs from the loss-of-consciousness estimation apparatus 2 in that it includes a control unit 20a instead of the control unit 20.

The control unit 20a differs from the control unit 20 in that it further executes grayout determination processing in addition to the out-of-range data determination processing, the ventricular state estimation processing, the loss-of-consciousness probability acquisition processing, and the loss-of-consciousness determination processing. The grayout determination processing is executed before the execution of the ventricular state estimation processing. The grayout determination processing may also be executed before the execution of the out-of-range data determination processing, or may be executed after the execution of the out-of-range data determination processing.

The grayout determination processing is processing for determining whether or not a condition relating to grayout (hereinafter referred to as a “grayout condition”) is satisfied based on the cerebral blood flow correlation amount time series.

The grayout condition is the condition that the probability that grayout will occur in the estimation target 901 exceeds a predetermined probability. Grayout is a phenomenon that signals loss of consciousness, and occurs before loss of consciousness occurs. More specifically, the grayout condition is the condition that there is data in which the cerebral blood flow correlation amount in each piece of cerebral blood flow correlation amount point data is less than a predetermined threshold value. The loss-of-consciousness estimation system 100 transmits information indicating that there is a high probability that grayout will occur (hereinafter referred to as a “grayout warning”) to the transmission destination if the grayout condition is satisfied.

Hereinafter, for the sake of simplicity of description, functional units having the same functions as those included in the loss-of-consciousness estimation system 100 are denoted by the same reference numerals as in FIG. 3, and description thereof is omitted.

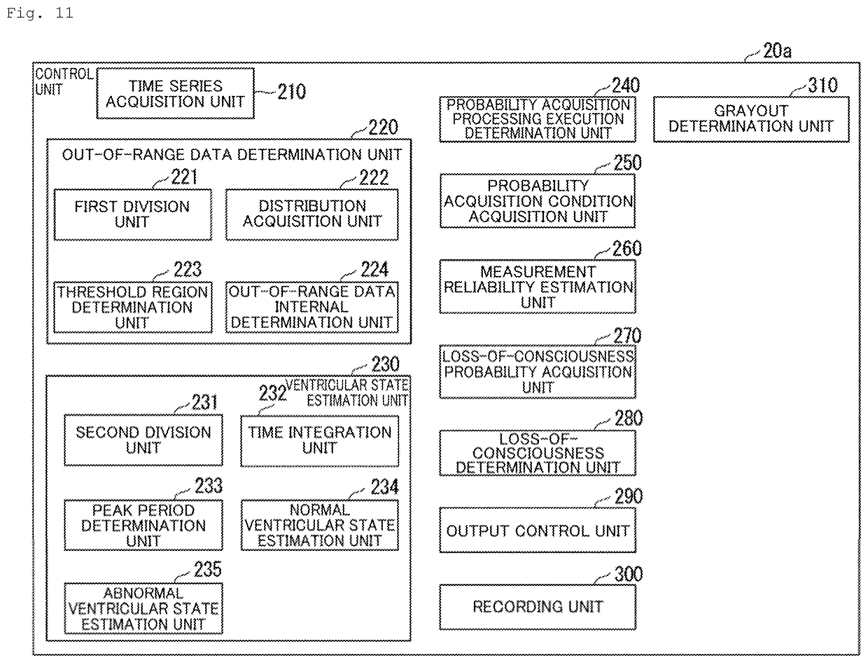

FIG. 11 is a diagram showing an example of a functional configuration of the control unit 20a in the second embodiment. The control unit 20a differs from the control unit 20 in that it includes a grayout determination unit 310. The grayout determination unit 310 executes the grayout determination processing. The determination result of the grayout determination processing is output by the output unit 24 due to the control of the output control unit 290. Hereinafter, for the sake of simplicity of description, functional units having the same functions as those included in the control unit 20 are denoted by the same reference numerals as those in FIG. 4, and description thereof is omitted.

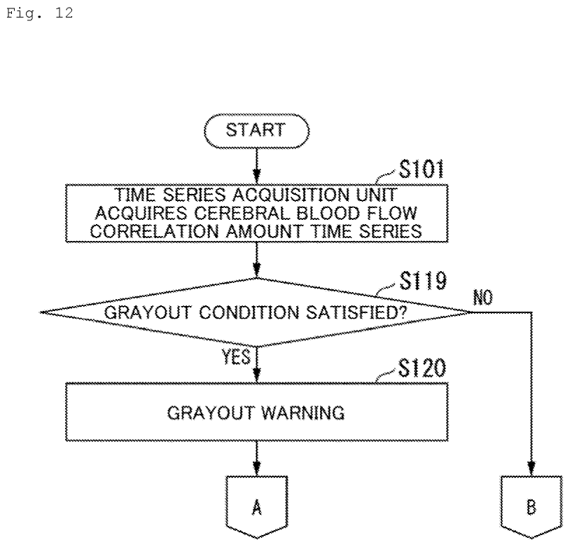

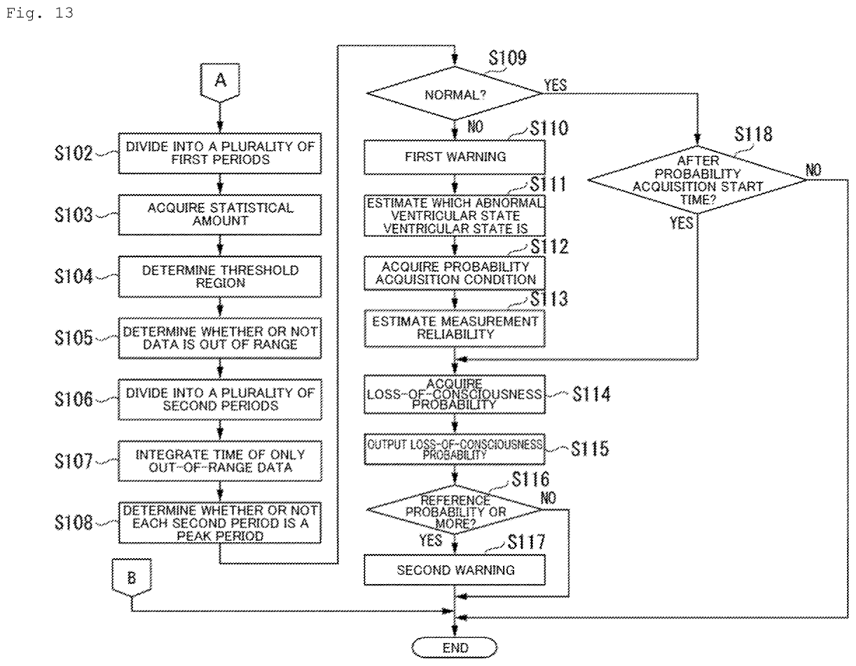

Hereinafter, an example of a flow of processing executed by the loss-of-consciousness estimation system 100a will be shown with reference to FIGS. 12 and 13. Hereinafter, for the sake of simplicity of description, the same processing as that shown in the flowchart of FIG. 5 is denoted by the same reference numerals as those n FIG. 5, and description thereof is omitted.

FIG. 12 is a first flowchart showing an example of a flow of processing executed by the loss-of-consciousness estimation system 100a of the second embodiment. FIG. 13 is a second flowchart showing an example of a flow of processing executed by the loss-of-consciousness estimation system 100a of the second embodiment.

Following step S101, the grayout determination unit 310 executes the grayout determination processing (step S119). Specifically, the grayout determination unit 310 determines whether or not the grayout condition is satisfied based on the cerebral blood flow correlation amount time series acquired in step S101.

If the grayout determination condition is satisfied (step S119: YES), the output control unit 290 causes the output unit 24 to output a grayout warning (step S120). Next, the processing of step S102 is executed. On the other hand, if the grayout determination condition is not satisfied (step S119: NO), the processing in the unit period ends.

The loss-of-consciousness estimation system 100a of the second embodiment configured in this manner further includes a function of determining the probability of grayout occurring in addition to the function of the loss-of-consciousness estimation system 100. For this reason, the loss-of-consciousness estimation system 100a of the second embodiment configured in this manner can reduce the danger posed by loss of consciousness.

### Third Embodiment

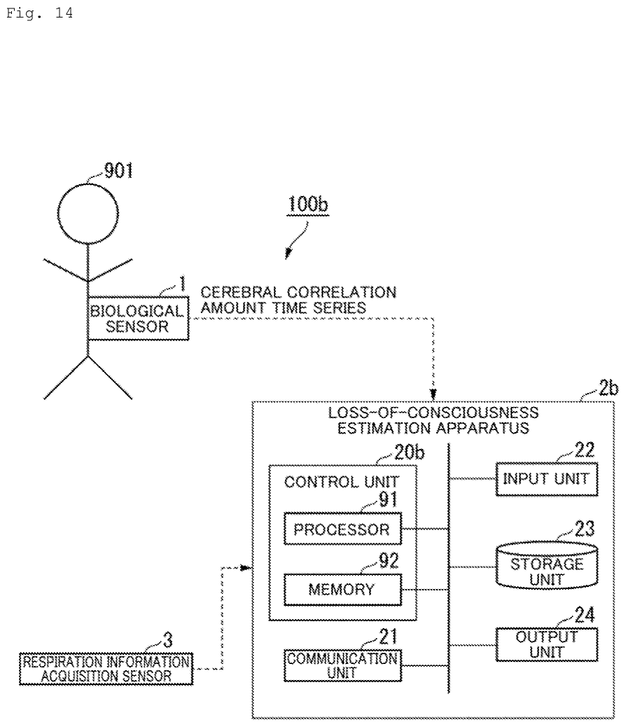

FIG. 14 is a diagram showing an example of a system configuration of a loss-of-consciousness estimation system 100b of a third embodiment. The loss-of-consciousness estimation system 100b differs from the loss-of-consciousness estimation system 100a in that it includes a respiration information acquisition sensor 3 and in that it includes a loss-of-consciousness estimation apparatus 2b instead of the loss-of-consciousness estimation apparatus 2a.

The respiration information acquisition sensor 3 acquires information related to the respiration of the estimation target 901 (hereinafter referred to as “respiration information”). The respiration information is, for example, information indicating whether the respiratory state of the estimation target 901 is in an expiratory phase or an inspiratory phase. The respiration information acquisition sensor 3 is, for example, a device that measures oxygen saturation. The respiration information acquisition sensor 3 may also be a device that measures the amount of ventilation. The respiration information acquisition sensor 3 may also be a device that measures the carbon dioxide concentration in the exhaled breath.

The loss-of-consciousness estimation apparatus 2b differs from the loss-of-consciousness estimation apparatus 2a in that it includes a control unit 20b instead of the control unit 20a. The control unit 20b differs from the control unit 20a in that it further executes grayout condition determination processing in addition to the grayout determination processing, the out-of-range data determination processing, the ventricular state estimation processing, the loss-of-consciousness probability acquisition processing, and the loss-of-consciousness determination processing. The grayout condition determination processing is processing for determining the grayout condition based on the respiration information.

The storage unit 23 in the third embodiment stores information indicating the correspondence relationship between the respiration information and the grayout condition (hereinafter referred to as “grayout condition correspondence information”) in advance. The grayout condition indicated by the grayout condition correspondence information may be any condition as long as the condition that there is cerebral blood flow correlation amount point data in which the cerebral blood flow correlation amount is less than a predetermined value (hereinafter referred to as “grayout threshold value”) is satisfied.

The correspondence relationship indicated by the grayout condition correspondence information is, for example, a relationship in which the grayout threshold value is increased when the respiration information indicates a decrease in the respiratory function, such as a decrease in oxygen saturation, a low ventilation amount, and a decrease in exhaled carbon dioxide concentration.

The communication unit 21 in the third embodiment is constituted by further including a communication interface for connecting to the respiration information acquisition sensor 3 in addition to the communication interface included in the communication unit 21 in the second embodiment. The communication unit 21 in the third embodiment communicates with the respiration information acquisition sensor 3 via, for example, a network. The communication unit 21 in the third embodiment acquires respiration information from the respiration information acquisition sensor 3 by communicating with the respiration information acquisition sensor 3.

Hereinafter, for the sake of simplicity of description, functional units having the same functions as those included in the loss-of-consciousness estimation system 100a are denoted by the same reference numerals as those in FIG. 10, and description thereof is omitted.

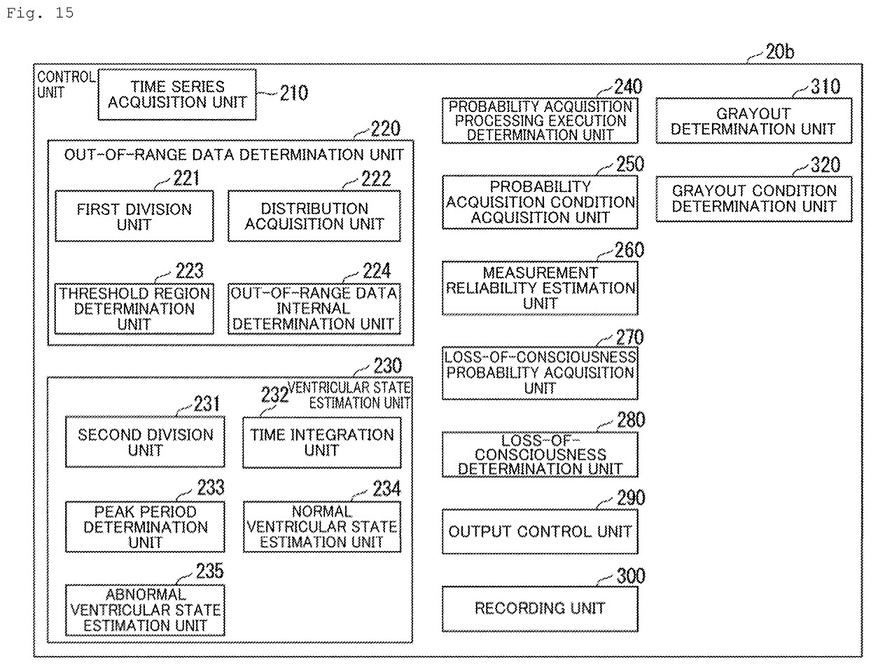

FIG. 15 is a diagram showing an example of the functional configuration of the control unit 20b according to the third embodiment. The control unit 20b differs from the control unit 20a in that it includes a grayout condition determination unit 320. Hereinafter, functional units having the same functions as those of the control unit 20a are denoted by the same reference numerals as those in FIG. 11 and description thereof is omitted.

The grayout condition determination unit 320 executes the grayout condition determination processing. More specifically, the grayout condition determination processing is processing for acquiring the grayout condition corresponding to the acquired respiration information using the grayout condition correspondence information and determining the acquired grayout condition as the grayout condition to be used in the grayout determination processing.

The grayout condition determination processing is executed before the grayout determination processing is executed in each unit period. For this reason, the grayout condition determination processing is executed, for example, after the processing of step S101 in FIG. 12 and before the processing of step S119 is executed.

The loss-of-consciousness estimation system 100b of the third embodiment configured in this manner determines the grayout condition based on the respiration information. For this reason, the loss-of-consciousness estimation system 100b can even further improve the accuracy of determining grayout compared to the loss-of-consciousness estimation system 100a. For this reason, the loss-of-consciousness estimation system 100b of the third embodiment configured in this manner can further reduce the danger posed by loss of consciousness.

### Fourth Embodiment

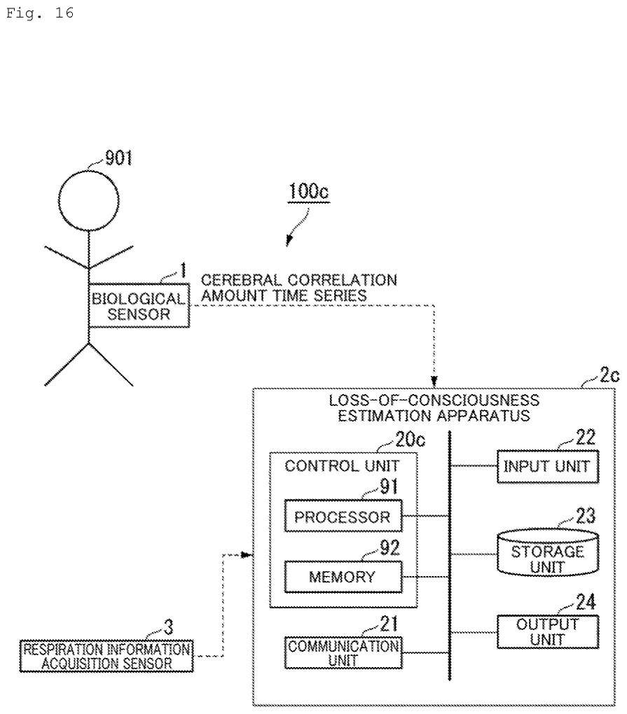

FIG. 16 is a diagram showing an example of a system configuration of a loss-of-consciousness estimation system 100c of a fourth embodiment. The loss-of-consciousness estimation system 100c differs from the loss-of-consciousness estimation system 100b in that it includes a loss-of-consciousness estimation apparatus 2c instead of the loss-of-consciousness estimation apparatus 2b.

The loss-of-consciousness estimation apparatus 2c differs from the loss-of-consciousness estimation apparatus 2b in that it includes a control unit 20c instead of the control unit 20b. The control unit 20c differs from the control unit 20b in that it further executes probability determination condition candidate acquisition processing in addition to the grayout condition determination processing, the grayout determination processing, the out-of-range data determination processing, the ventricular state estimation processing, the loss-of-consciousness probability acquisition processing, and the loss-of-consciousness determination processing.

The probability determination condition candidate acquisition processing is processing for determining a candidate for a probability determination condition based on respiration information. Specifically, a candidate for a probability determination condition is information indicating the relationship between the ventricular state and the probability determination condition.

The storage unit 23 in the fourth embodiment stores information indicating the correspondence relationship between the respiration information and the candidate for the probability determination condition (hereinafter referred to as “probability determination condition candidate correspondence information”) in advance.

The correspondence relationship indicated by the probability determination condition candidate correspondence information is a relationship in which, for example, if the respiration information indicates a decrease in respiratory function, the amount of change per unit time in the amount of change depending on the elapsed time of the loss-of-consciousness probability is larger than usual.

The communication unit 21 in the fourth embodiment is the same as the communication unit 21 in the third embodiment.

Hereinafter, for the sake of simplicity of description, functional units having the same functions as those included in the loss-of-consciousness estimation system 100b are denoted by the same reference numerals as those in FIG. 14, and description thereof is omitted.

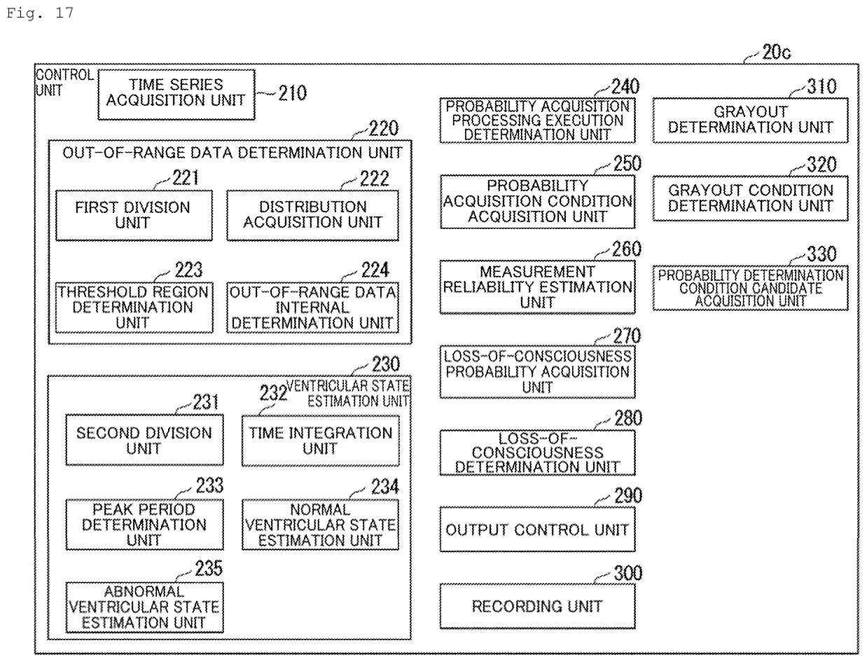

FIG. 17 is a diagram showing an example of a functional configuration of the control unit 20c according to the fourth embodiment. The control unit 20c differs from the control unit 20b in that it includes a probability determination condition candidate acquisition unit 330. Hereinafter, units having the same function as those of the control unit 20b are denoted by the same reference numerals as those in FIG. 15, and description thereof is omitted.

The probability determination condition candidate acquisition unit 330 executes the probability determination condition candidate acquisition processing. More specifically, the probability determination condition candidate acquisition processing is processing for acquiring a candidate for a probability determination condition corresponding to the acquired respiration information using the probability determination condition candidate correspondence information. From among the acquired candidates for the probability determination condition, the probability acquisition condition acquisition unit 250 acquires the probability determination condition in the loss-of-consciousness probability acquisition processing based on the estimation result of the ventricular state estimation unit 230.

The probability determination condition candidate acquisition processing is executed before the probability acquisition condition acquisition unit 250 acquires the probability acquisition condition in each unit period. For this reason, the probability determination condition candidate acquisition processing may also be executed at any timing as long as it is executed after the execution of step S101 in FIG. 12 and before the execution of the processing of step S112 in FIG. 13.

The loss-of-consciousness estimation system 100c of the fourth embodiment configured in this manner further estimates the loss-of-consciousness probability of the estimation target 901 based also on respiration information, in addition to the information used by the loss-of-consciousness estimation system 100b for estimating the loss-of-consciousness probability. For this reason, the loss-of-consciousness estimation system 100c can improve the accuracy of estimating the likelihood of loss of consciousness of the estimation target 901 even more than the loss-of-consciousness estimation system 100b. For this reason, the loss-of-consciousness estimation system 100c of the fourth embodiment configured in this manner can further reduce the danger posed by loss of consciousness compared to the loss-of-consciousness estimation system 100b.

### Fifth Embodiment

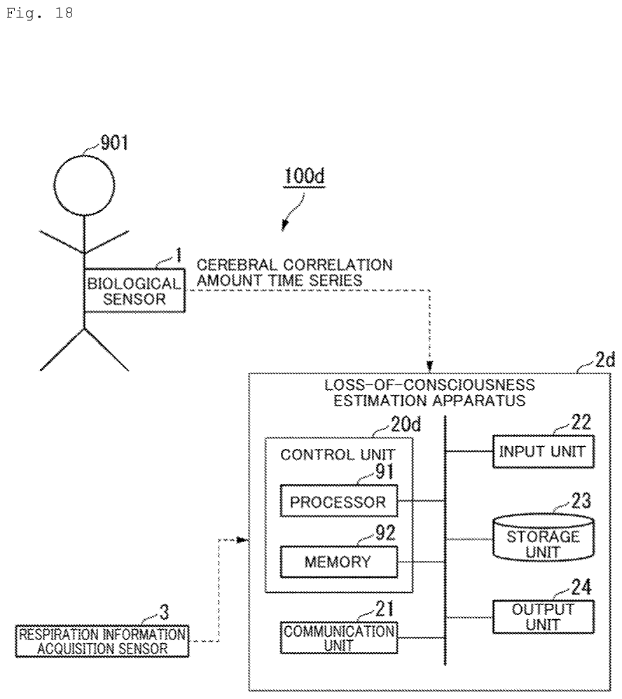

FIG. 18 is a diagram showing an example of a system configuration of a loss-of-consciousness estimation system 100d according to a fifth embodiment. The loss-of-consciousness estimation system 100d differs from the loss-of-consciousness estimation system 100c in that it includes a loss-of-consciousness estimation apparatus 2d instead of the loss-of-consciousness estimation apparatus 2c.

The loss-of-consciousness estimation apparatus 2d differs from the loss-of-consciousness estimation apparatus 2c in that it includes a control unit 20d instead of the control unit 20c. The control unit 20d differs from the control unit 20c in that it further executes ventricular state estimation condition determination processing in addition to the processing executed by the control unit 20c. Specifically, the processing executed by the control unit 20c is the grayout condition determination processing, the grayout determination processing, the out-of-range data determination processing, the ventricular state estimation processing, the probability determination condition candidate acquisition processing, the loss-of-consciousness probability acquisition processing, and the loss-of-consciousness determination processing.

The ventricular state estimation condition determination processing is processing for determining a condition (hereinafter referred to as “ventricular state estimation condition”) to be used for the ventricular state estimation processing based on respiration information.

The ventricular state estimation conditions are, for example, a first length, and a threshold region upper limit value and threshold value region lower limit value at each time. In such a case, the ventricular state estimation condition determination processing is processing for changing the first length and the threshold region upper limit value and threshold value area lower limit value at each time according to the respiration information. That is, an example of the ventricular state estimation condition determination processing is processing for determining the first length according to the respiration information and the threshold region upper limit value and threshold value area lower limit value at each time.

The ventricular state estimation condition is, for example, information indicating a first reference frequency or a second reference frequency. In such a case, the ventricular state estimation condition determination processing is, for example, processing for changing the first reference frequency or the second reference frequency according to the respiration information. In other words, an example of the ventricular state estimation condition determination processing is processing for determining the first reference frequency or the second reference frequency according to the respiration information. In the ventricular state estimation condition determination processing, for example, the first reference frequency and the second reference frequency may be adjusted according to the respiratory state, such as a decrease in respiratory function or respiratory arrest.

The storage unit 23 in the fifth embodiment stores information indicating the correspondence relationship between the respiration information and the ventricular state estimation condition (hereinafter referred to as “ventricular state estimation condition correspondence information”) in advance.

The correspondence relationship indicated by the ventricular state estimation condition correspondence information is, for example, a relationship in which a significant ventricular abnormality, ventricular fibrillation or cardiac arrest, or an imminent situation thereof is obtained when the respiration information indicates a respiratory arrest state for 20 seconds or more continuously.

The communication unit 21 in the fifth embodiment is the same as the communication unit 21 in the third embodiment.

Hereinafter, for the sake of simplicity of description, functional units having the same functions as those included in the loss-of-consciousness estimation system 100d are denoted by the same reference numerals as those in FIG. 17, and description thereof is omitted.

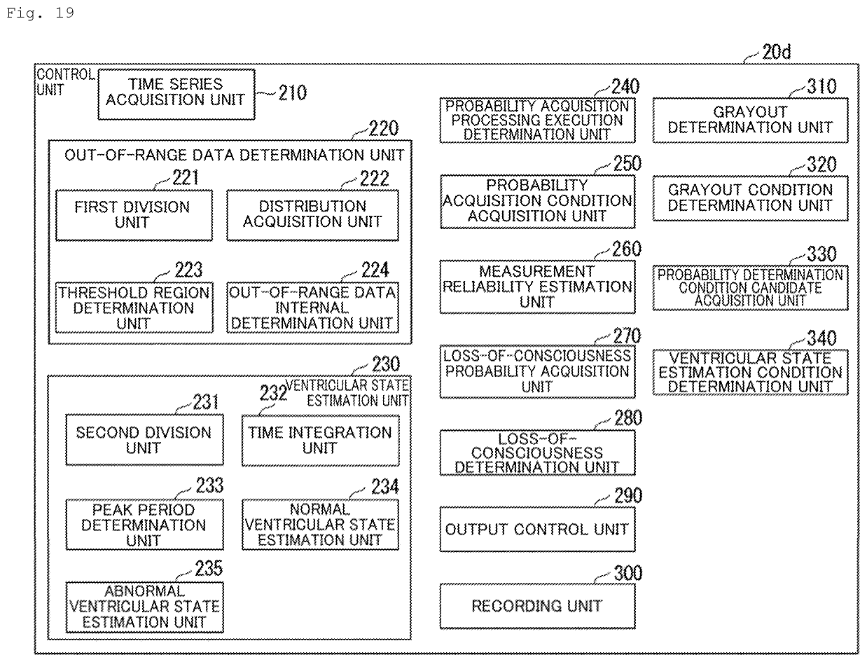

FIG. 19 is a diagram showing an example of a functional configuration of the control unit 20d according to the fifth embodiment. The control unit 20d differs from the control unit 20c in that it includes a ventricular state estimation condition determination unit 340. Hereinafter, units having the same functions as the control unit 20c are denoted by the same reference numerals as those in FIG. 17, and description thereof is omitted. The ventricular state estimation condition determination unit 340 executes the ventricular state estimation condition determination processing. The ventricular state estimation condition determination unit 340 determines the ventricular state estimation condition corresponding to the acquired respiration information, based on the ventricular state estimation condition correspondence information and the acquired respiration information by executing the ventricular state estimation condition determination processing.

The ventricular state estimation condition determination processing is executed in each unit period before the ventricular state estimation unit 230 starts estimating the ventricular state. For this reason, the ventricular state estimation condition determination processing may be executed at any timing as long as it is executed after the execution of step S101 in FIG. 12 and before the execution of the processing of step S103 in FIG. 13.

The loss-of-consciousness estimation system 100d of the fifth embodiment configured in this manner determines the ventricular state estimation condition based on the respiration information. For this reason, the loss-of-consciousness estimation system 100d can improve the accuracy of estimating the likelihood of loss of consciousness of the estimation target 901 even more than the loss-of-consciousness estimation system 100c. For this reason, the loss-of-consciousness estimation system 100d of the fifth embodiment configured in this manner can further reduce the danger posed by loss of consciousness compared to the loss-of-consciousness estimation system 100c.

### Sixth Embodiment

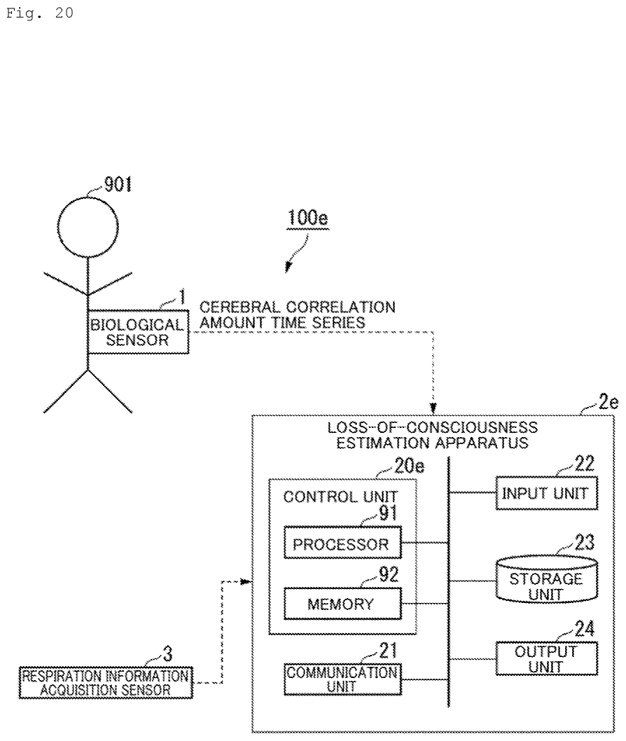

FIG. 20 is a diagram showing an example of a system configuration of a loss-of-consciousness estimation system 100e of a sixth embodiment. The loss-of-consciousness estimation system 100e differs from the loss-of-consciousness estimation system 100d in that it includes a loss-of-consciousness estimation apparatus 2e instead of the loss-of-consciousness estimation apparatus 2d.

The loss-of-consciousness estimation apparatus 2e differs from the loss-of-consciousness estimation apparatus 2d in that it includes a control unit 20e instead of the control unit 20d. The control unit 20e differs from the control unit 20d in that it further executes learning update processing in addition to the processing executed by the control unit 20d.

Specifically, the processing executed by the control unit 20e is the grayout condition determination processing, the grayout determination processing, the out-of-range data determination processing, the ventricular state estimation condition determination processing, the ventricular state estimation processing, the probability determination condition candidate acquisition processing, the loss-of-consciousness probability acquisition processing and the loss-of-consciousness determination processing.

The learning update processing includes learning processing and update processing. The learning processing is processing for learning conditions used for estimation such as the grayout condition, the ventricular state estimation condition, the probability determination condition, and the reference probability through machine learning based on a user's response to the output estimation result. The update processing is processing for updating the conditions used for estimation based on the learning result of the learning processing.

The output estimation result is the information output from the output unit 24 and is information indicating the estimation result of the loss-of-consciousness estimation apparatus 2e related to the loss of consciousness. The output estimation result is, for example, the grayout warning, the first warning, the loss-of-consciousness probability, or the second warning.

The user's response to the output estimation result means that the user inputs the certainty of the output estimation result to the loss-of-consciousness estimation apparatus 2e. The user's response is input via, for example, the input unit 22. The user's response may also be input via the communication unit 21. Note that the user is the estimation target 901 or the manager 902.

Hereinafter, for the sake of simplicity of description, functional units having the same function as those included in the loss-of-consciousness estimation system 100d are denoted by the same reference numerals as those in FIG. 18, and description thereof is omitted.

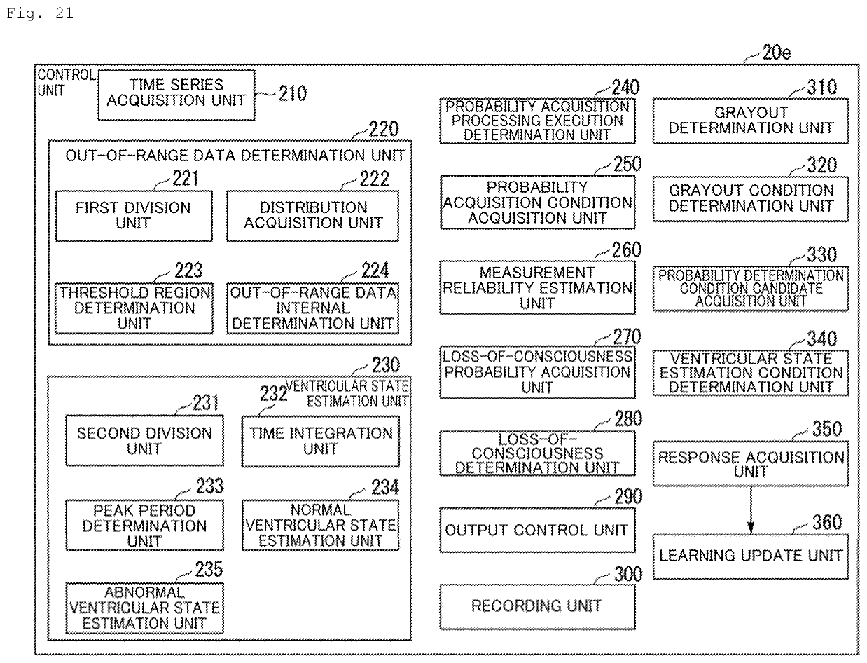

FIG. 21 is a diagram showing an example of a functional configuration of the control unit 20e according to the sixth embodiment. The control unit 20e differs from the control unit 20d in that it includes a response acquisition unit 350 and a learning update unit 360. Hereinafter, units having the same functions as those of the control unit 20d are denoted by the same reference numerals as those in FIG. 19, and description thereof is omitted.

The response acquisition unit 350 acquires the user's response input via the input unit 22 or the communication unit 21. The learning update unit 360 executes the learning update processing. Specifically, the learning update unit 360 first learns the conditions used for estimation based on the user's response acquired by the response acquisition unit 350, then updates the conditions used for estimation based on the learning result.

The learning update processing may also be executed at any timing as long as the storage unit 23 stores the correspondence relationship between the output estimation result and the user's response.

The loss-of-consciousness estimation system 100e of the sixth embodiment configured in this manner updates the conditions used for estimation based on the user's response to the output estimation result. For this reason, the loss-of-consciousness estimation system 100e can improve the accuracy of estimating the likelihood of loss of consciousness of the estimation target 901 even more than the loss-of-consciousness estimation system 100d. For this reason, the loss-of-consciousness estimation system 100e of the fifth embodiment configured in this way can further reduce the danger posed by loss of consciousness compared to the loss-of-consciousness estimation system 100d.

(First Variation)

The threshold region may have only one of the lower threshold value and the upper threshold value, as long as the ventricular state estimation unit 230 can determine whether or not the ventricular state of the estimation target 901 is an abnormal ventricular state. For example, if the abnormal ventricular state is a ventricular extrasystole, the electrode lead for measuring the cardiac potential is fixed, and the method of excitatory propagation of the extrasystole is constant, the threshold region may be, for example, only the upper threshold value.

Note that the threshold region having only the upper threshold value means that the range indicated by the threshold region is a range including all values greater than or equal to the upper threshold value. Note that the threshold region having only the lower threshold value means that the range indicated by the threshold region is a range including all values greater than or equal to the lower threshold value.

If the abnormal ventricular state is a ventricular extrasystole, the depolarization time of the ventricle is longer than normal. For this reason, it is often the case that the potential remains outside of the threshold value for a long time, and that the electrode guidance for measuring the cardiac potential is fixed at a constant position and the direction of excitatory propagation of the extrasystole is constant. For this reason, even if the threshold region has only the upper threshold value or only the lower threshold value, the ventricular state estimation unit 230 can determine whether or not the ventricular state of the estimation target 901 is a ventricular extrasystole.

For example, if the threshold region has only the upper threshold value, the out-of-range data internal determination unit 224 determines whether or not the cerebral blood flow correlation amount exceeds the upper threshold value for each piece of cerebral blood flow correlation amount point data of the cerebral blood flow correlation amount time series, and does not determine whether or not the cerebral blood flow correlation amount is less than the lower threshold value. In such a case, the out-of-range data includes the cerebral blood flow correlation amount point data in which the cerebral blood flow correlation amount exceeds the upper threshold value and does not include the cerebral blood flow correlation amount point data in which the cerebral blood flow correlation amount is less than the lower threshold value.

For example, if the threshold region has only the lower threshold, the out-of-range data internal determination unit 224 determines whether or not the cerebral blood flow correlation amount is less than the lower threshold value for each piece of cerebral blood flow correlation amount point data of the cerebral blood flow correlation amount time series, and does not determine whether or not the cerebral blood flow correlation amount exceeds the upper threshold value. In such a case, the out-of-range data includes the cerebral blood flow correlation amount point data in which the cerebral blood flow correlation amount is less than the lower threshold value and does not include the cerebral blood flow correlation amount point data in which the cerebral blood flow correlation amount exceeds the upper threshold value.

Even if the threshold region has a lower threshold value and an upper threshold value, if the ventricular state estimation unit 230 can estimate whether or not the ventricular state of the estimation target 901 is an abnormal ventricular state, the out-of-range data internal determination unit 224 does not necessarily need to determine out-of-range data using the lower threshold value and the upper threshold value.

If the ventricular state estimation unit 230 can estimate whether or not the ventricular state of the estimation target 901 is an abnormal ventricular state, the out-of-range data internal determination unit 224 may determine out-of-range data using, for example, only the upper threshold value. In such a case, the out-of-range data internal determination unit 224 determines whether or not the cerebral blood flow correlation amount exceeds the upper threshold value for each piece of cerebral blood flow correlation amount point data of the cerebral blood flow correlation amount time series, and does not determine whether or not the blood flow correlation amount is less than the lower threshold value. In such a case, the cerebral blood flow correlation amount point data in which the cerebral blood flow correlation amount exceeds the upper threshold value is cerebral blood flow correlation amount point data that is out of range of the threshold region, and the cerebral blood flow correlation amount point data in which the cerebral blood flow correlation amount is less than the lower threshold value is cerebral blood flow correlation amount point data within the range of the threshold region.

If the ventricular state estimation unit 230 can determine whether or not the ventricular state of the estimation target 901 is an abnormal ventricular state, the out-of-range data internal determination unit 224 may determine out-of-range data using, for example, only the lower threshold value. In such a case, the out-of-range data internal determination unit 224 determines whether or not the cerebral blood flow correlation amount is less than the lower threshold value for each piece of cerebral blood flow correlation amount point data of the cerebral blood flow correlation amount time series, and does not determine whether or not the cerebral blood flow correlation amount exceeds the upper threshold value. In such a case, the cerebral blood flow correlation amount point data in which the cerebral blood flow correlation amount is less than the lower threshold value is the cerebral blood flow correlation amount point data outside the range of the threshold region, and the cerebral blood flow correlation amount point data in which the cerebral blood flow correlation amount exceeds the upper threshold value is the cerebral blood flow correlation amount point data within the range of the threshold region.

Thus, if the ventricular state estimation unit 230 can estimate whether or not the ventricular state of the estimation target 901 is an abnormal ventricular state, the out-of-range data internal determination unit 224 does not necessarily need to determine the out-of-range data using both the upper threshold value and the lower threshold value.

The loss-of-consciousness estimation systems 100, and 100a to 100e of the first variation configured in this manner determine the threshold region corresponding to the position in the time axis direction of each piece of data, based on data within a predetermined period including the position in the time axis direction of each piece of data in the cerebral blood flow correlation amount time series. Then, the loss-of-consciousness estimation systems 100 and 100a to 100e estimate the loss-of-consciousness probability based on the determined threshold value region. For this reason, the loss-of-consciousness estimation systems 100 and 100a to 100e can improve the accuracy of estimating the likelihood of loss of consciousness of the estimation target 901. Also, for this reason, the loss-of-consciousness estimation systems 100 and 100a to 100e can improve the accuracy of estimating the probability that consciousness has already been lost.

Also, the loss-of-consciousness estimation systems 100 and 100a to 100e of the first variation configured in this manner output the loss-of-consciousness probability that is the estimation result using the output unit 24. Since the loss-of-consciousness probability is output, the estimation target 901 can take actions to reduce the danger posed by loss of consciousness before loss of consciousness occurs. For this reason, the loss-of-consciousness estimation systems 100 and 100a to 100e of the first variation configured in this manner can reduce the danger posed by loss of consciousness.

(Second Variation)

The control units 20 and 20a to 20e may execute signal shaping processing before executing one of the out-of-range data determination processing and the grayout determination processing, which is executed first. The signal shaping processing is processing for shaping the cerebral blood flow correlation amount time series so as to be suitable for the subsequent processing. Shaping to be suitable means shaping to be a series that satisfies a predetermined condition determined in advance. The signal shaping processing is, for example, processing (high-pass processing) for removing high-frequency components included in the cerebral blood flow correlation amount time series, such as noise generated when a biological signal is acquired, from the cerebral blood flow correlation amount time series. The signal shaping processing may be, for example, processing for suppressing fluctuation of the baseline of the biological signal. The signal shaping processing may be, for example, processing for normalizing the potential width of the biological signal.

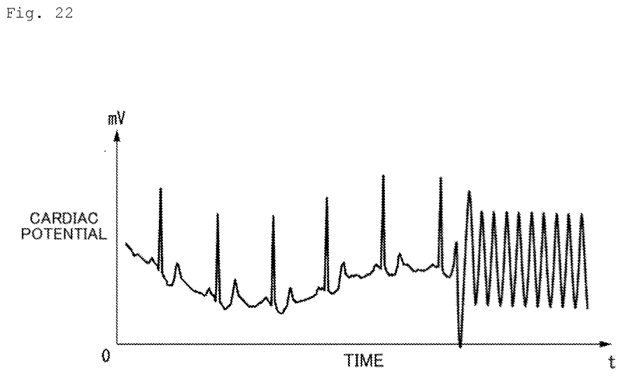

FIG. 22 is a diagram showing an example of a cardiac potential time series before shaping in the second variation. The horizontal axis of the graph in FIG. 22 shows the elapsed time from the time of the origin. The vertical axis of FIG. 22 shows the potential of the cardiac potential. In the cardiac potential time series shown in FIG. 22, fluctuations are observed in the cardiac potential. For this reason, if the loss-of-consciousness probability is acquired using the data as it is, the accuracy of the acquisition result may be low.

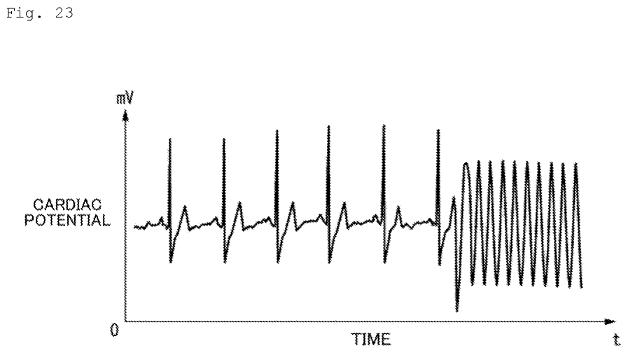

FIG. 23 is a diagram showing an example of the cardiac potential time series after shaping by the high-pass filter processing in the second variation. The horizontal axis of the graph in FIG. 23 shows the elapsed time from the time of the origin. The vertical axis of FIG. 23 shows the potential of the cardiac potential. The graph of FIG. 23 shows the execution result of the signal shaping processing for the cardiac potential time series shown in FIG. 22. In FIG. 23, fluctuation in the baseline of the cardiac potential is suppressed compared to the cardiac potential time series of FIG. 22. For this reason, if the loss-of-consciousness probability is acquired using the cardiac potential time series of FIG. 23, the accuracy of the acquisition result is higher than in the case where the loss-of-consciousness probability is acquired using the cardiac potential time series of FIG. 22.

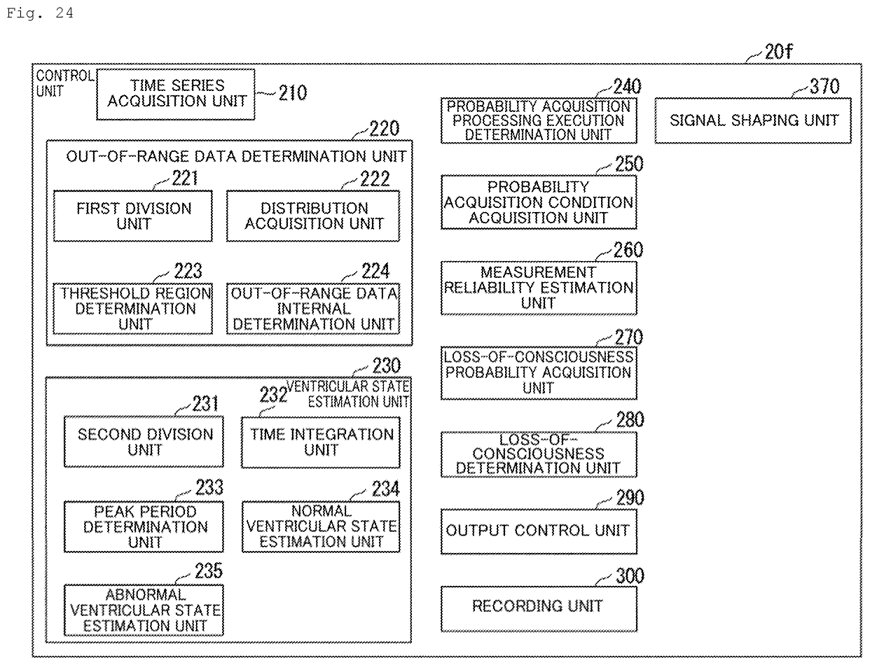

FIG. 24 is a diagram showing an example of a functional configuration of a control unit 20 (hereinafter referred to as “control unit 20f”) that executes the signal shaping processing as an example of the control units 20 and 20a to 20e that execute the signal shaping processing in the second variation.

The control unit 20f differs from the control unit 20 in that it includes a signal shaping unit 370. The signal shaping unit 370 executes signal shaping processing on the cerebral blood flow correlation amount time series acquired by the time series acquisition unit 210. In such a case, the cerebral blood flow correlation amount time series used in each process executed after the execution of the signal shaping processing, such as the out-of-range data determination processing, the ventricular state estimation processing, the loss-of-consciousness probability acquisition processing, and the loss-of-consciousness determination processing, is a shaped cerebral blood flow correlation amount time series. If the control unit 20f executes the grayout determination processing, each process executed after the execution of the signal shaping processing also includes the grayout determination processing.

Specifically, the signal shaping processing is executed after the execution of the processing of step S101 and before the execution of the processing of step S119. Also, if the processing of step S119 is not executed, the signal shaping processing is executed after the execution of the processing of step S101 and before the execution of the processing of step S102.

The loss-of-consciousness estimation systems 100 and 100a to 100e of the second variation configured in this manner execute the loss-of-consciousness probability acquisition processing based on the shaped cerebral blood flow correlation amount time series. For this reason, the loss-of-consciousness estimation systems 100 and 100a to 100e of the second variation can improve the accuracy of estimating the likelihood of loss of consciousness of the estimation target 901. For this reason, the loss-of-consciousness estimation systems 100 and 100a to 100e of the second variation configured in this manner can reduce the danger posed by loss of consciousness.

Also, the loss-of-consciousness estimation systems 100a to 100e of the second variation configured in this manner execute the out-of-range data determination processing, the ventricular state estimation processing, the loss-of-consciousness probability acquisition processing, the loss-of-consciousness determination processing, and the grayout determination processing based on the shaped cerebral blood flow correlation amount time series. For this reason, the loss-of-consciousness estimation systems 100a to 100e of the second variation configured in this manner can reduce the danger posed by loss of consciousness.

(Third Variation)

The control units 20 and 20a to 20e may execute signal modeling processing before executing one of the out-of-range data determination processing and the grayout determination processing, which is executed first. In the signal modeling processing, a waveform based on a predetermined theory relating to the cerebral blood flow correlation amount, which is a waveform in which the difference from the cerebral blood flow correlation amount time series (hereinafter referred to as “model waveform”) is less than a predetermined difference, is acquired based on the cerebral blood flow correlation amount time series acquired by the time series acquisition unit 210. The predetermined theory that relates the cerebral blood flow correlation amount time series to the cerebral blood flow correlation amount is, for example, the Frank-Starling law, or a non-linear finite element model of cardiac circulation.

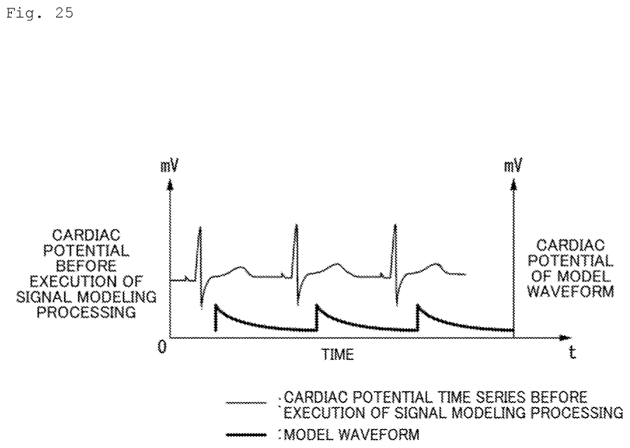

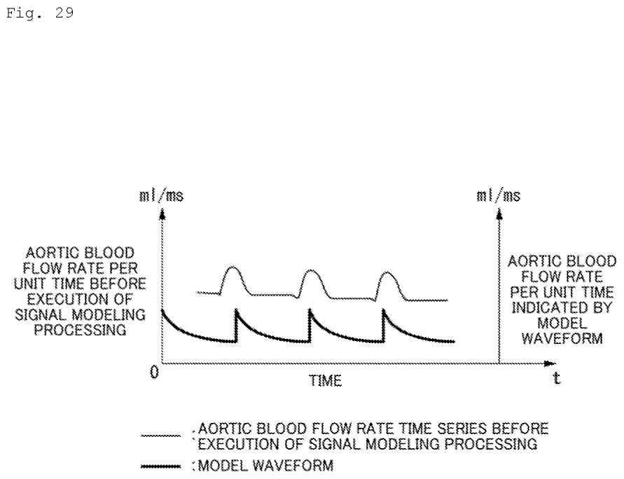

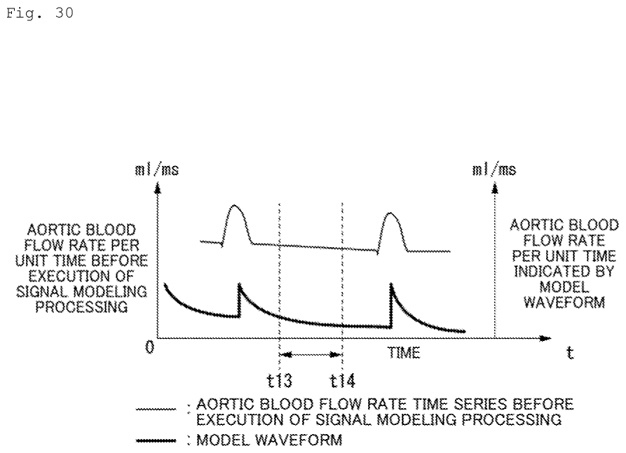

Here, an example of the correspondence between the cerebral blood flow correlation amount time series and the model waveform is shown using a two-axis graph with reference to FIGS. 25 to 30. Specifically, FIGS. 25 to 28 show the correspondence between the cardiac potential time series and the model waveform. FIGS. 29 and 30 show the correspondence between an aortic blood flow rate time series and the model waveform. The aortic blood flow rate time series is a time series of the aortic blood flow rate.

FIG. 25 is a first diagram showing the correspondence between the cardiac potential time series and the model waveform in the third variation. The horizontal axis of the graph in FIG. 25 shows the elapsed time from the time of the origin. The vertical axis on the left side of FIG. 25 shows the cardiac potential before executing the signal modeling processing. The vertical axis on the right side of FIG. 25 shows the cardiac potential of the model waveform. The cardiac potential time series before the execution of the signal modeling processing in FIG. 25 is a graph of the cardiac potential of the heart with normal movement. The model waveform of FIG. 25 is a model waveform acquired by executing the signal modeling processing based on the cardiac potential time series before the execution of the signal modeling processing of FIG. 25.

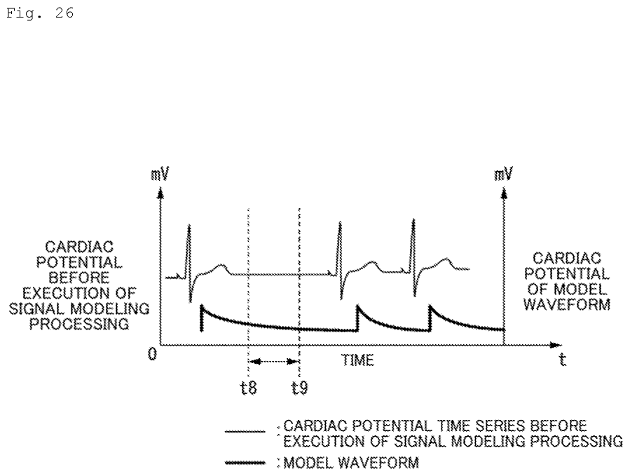

FIG. 26 is a second diagram showing the correspondence between the cardiac potential time series and the model waveform in the third variation. The horizontal axis of the graph in FIG. 26 shows the elapsed time from the time of the origin. The vertical axis on the left side of FIG. 26 shows the cardiac potential before the execution of the signal modeling processing. The vertical axis on the right side of FIG. 26 shows the cardiac potential of the model waveform. The cardiac potential time series before the execution of the signal modeling processing in FIG. 26 is a graph of the cardiac potential of the heart in which arrhythmia (bradyarrythmia) is occurring. The model waveform of FIG. 26 is a model waveform acquired by executing the signal modeling processing based on the cardiac potential time series before the execution of the signal modeling processing of FIG. 26. FIG. 26 shows that arrhythmia occurs during the period from time t8 to time t9.

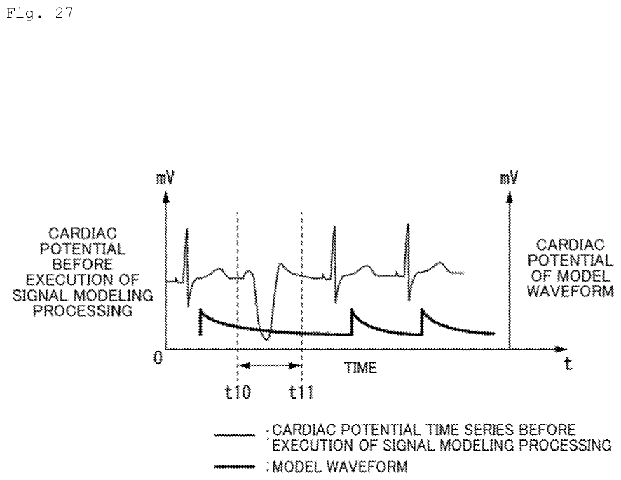

FIG. 27 is a third diagram showing the correspondence between the cardiac potential time series and the model waveform in the third variation. The horizontal axis of the graph in FIG. 27 shows the elapsed time from the time of the origin. The vertical axis on the left side of FIG. 27 shows the cardiac potential before the execution of the signal modeling processing. The vertical axis on the right side of FIG. 27 shows the cardiac potential of the model waveform. The cardiac potential time series before the execution of the signal modeling processing in FIG. 27 is a graph of the cardiac potential of a heart in which a ventricular extrasystole is occurring. The model waveform of FIG. 27 is a model waveform acquired by executing the signal modeling processing based on the cardiac potential time series before the execution of the signal modeling processing of FIG. 27. FIG. 27 shows that a ventricular extrasystole occurs during the period from time t10 to time t11.

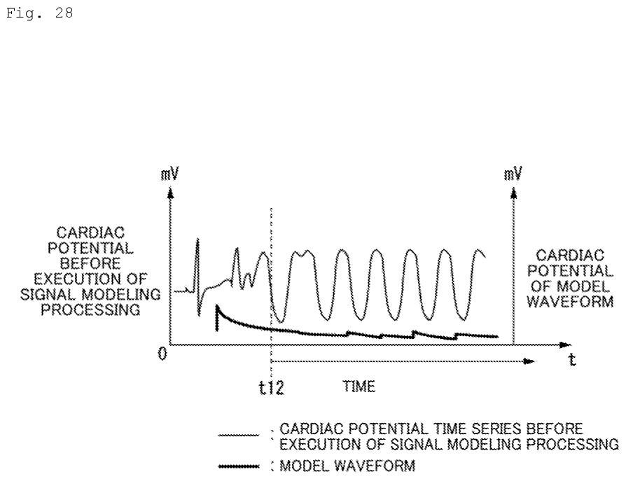

FIG. 28 is a fourth diagram showing the correspondence between the cardiac potential time series and the model waveform in the third variation. The horizontal axis of the graph in FIG. 28 shows the elapsed time from the time of the origin. The vertical axis on the left side of FIG. 28 shows the cardiac potential before the execution of the signal modeling processing. The vertical axis on the right side of FIG. 28 shows the cardiac potential of the model waveform. The cardiac potential time series before the execution of the signal modeling processing in FIG. 28 is a graph of the cardiac potential of a heart in which ventricular tachycardia is occurring. The model waveform of FIG. 28 is a model waveform acquired by executing the signal modeling processing based on the cardiac potential time series before the execution of the signal modeling processing of FIG. 28. FIG. 28 shows that ventricular tachycardia occurs at time t12 and onward.

FIG. 29 is a first diagram showing the correspondence between the aortic blood flow rate time series and the model waveform in the third variation. The horizontal axis of the graph in FIG. 29 shows the elapsed time from the time of the origin. The vertical axis on the left side of FIG. 29 shows the aortic blood flow rate per unit time before the execution of the signal modeling processing. The vertical axis on the right side of FIG. 29 shows the aortic blood flow rate per unit time indicated by the model waveform. The aortic blood flow rate time series before the execution of the signal modeling processing in FIG. 29 is a graph of the aortic blood flow rate with normal movement. The model waveform of FIG. 29 is a model waveform acquired by executing the signal modeling processing based on the aortic blood flow rate time series before the execution of the signal modeling processing of FIG. 29.

FIG. 30 is a second diagram showing the correspondence between the aortic blood flow rate time series and the model waveform in the third variation. The horizontal axis of the graph in FIG. 30 shows the elapsed time from the time of the origin. The vertical axis on the left side of FIG. 30 shows the aortic blood flow rate per unit time before executing the signal modeling processing. The vertical axis on the right side of FIG. 30 shows the aortic blood flow rate per unit time indicated by the model waveform. The aortic blood flow rate time series before the execution of the signal modeling processing in FIG. 30 is a graph of the aortic blood flow rate in which arrhythmia occurs. The model waveform of FIG. 30 is a model waveform acquired by executing the signal modeling processing based on the aortic blood flow rate time series before the execution of the signal modeling processing of FIG. 29. FIG. 30 shows that arrhythmia occurs during the period from time t13 to time t14. In the case of the aortic blood flow rate time series shown in FIG. 30, there is a high probability that the estimation target 901 will undergo loss of consciousness after time t14.

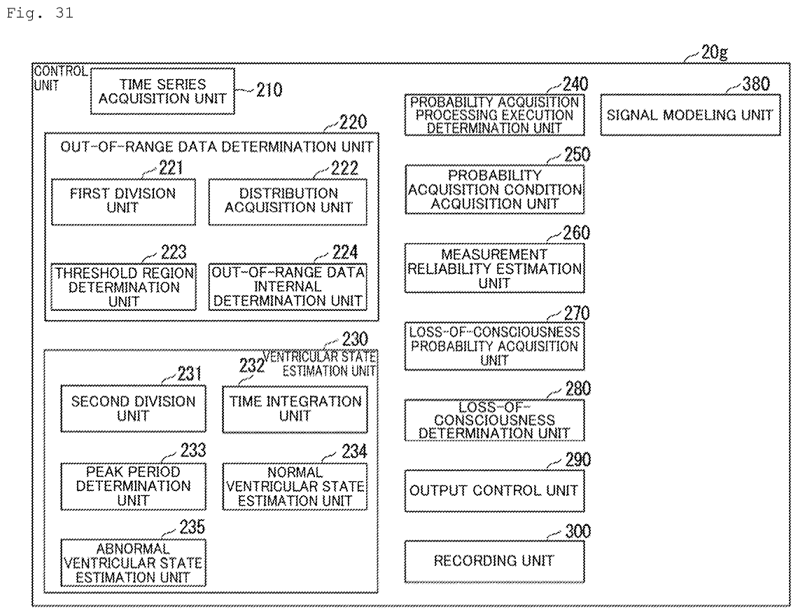

FIG. 31 is a diagram showing an example of a functional configuration of the control unit 20 (hereinafter referred to as “control unit 20g”) that executes the signal modeling processing as an example of the control units 20 and 20a to 20f that execute the signal modeling processing in the third variation.

The control unit 20g differs from the control unit 20 in that it includes a signal modeling unit 380. The signal modeling unit 380 executes signal modeling processing on the cerebral blood flow correlation amount time series acquired by the time series acquisition unit 210. In such a case, the cerebral blood flow correlation amount time series used in each process executed after the execution of signal shaping processing such as the out-of-range data determination processing, the ventricular state estimation processing, the loss-of-consciousness probability acquisition processing, and the loss-of-consciousness determination processing is a cerebral blood flow correlation amount time series resulting from the execution of the signal modeling processing. If the control unit 20g executes the grayout determination processing, each process executed after the execution of the signal modeling processing includes the grayout determination processing as well.

The loss-of-consciousness estimation systems 100 and 100a to 100e of the third variation configured in this manner execute the loss-of-consciousness probability acquisition processing based on the cerebral blood flow correlation amount time series modeled based on a theory. For this reason, the loss-of-consciousness estimation systems 100 and 100a to 100e of the third variation can improve the accuracy of estimating the likelihood of loss of consciousness of the estimation target 901. For this reason, the loss-of-consciousness estimation systems 100 and 100a to 100e of the third variation configured in this manner can reduce the danger posed by loss of consciousness.

Also, the loss-of-consciousness estimation systems 100a to 100e of the third variation configured in this manner execute out-of-range data determination processing, ventricular state estimation processing, loss-of-consciousness probability acquisition processing, loss-of-consciousness determination processing, and grayout determination processing based on the cerebral blood flow correlation amount time series modeled based on the theory. For this reason, the loss-of-consciousness estimation systems 100a to 100e of the third variation configured in this manner can reduce the danger posed by loss of consciousness.

(Fourth Variation)

The conditions used for estimation, such as the grayout condition, the ventricular state estimation condition, the probability determination condition, and the reference probability, may be conditions obtained based on the personal information of the estimation target 901. In such a case, the storage unit 23 stores, in advance, the conditions used for estimation, such as the grayout condition, the ventricular state estimation condition, the probability determination condition, and the reference probability, which have been adjusted in advance according to the estimation target 901. The personal information may be, for example, the age of the estimated target 901, gender, food preferences, height, weight, body fat percentage, or the presence or absence of an underlying disease such as arteriosclerosis or carotid artery stenosis.

(Fifth Variation)

Note that in the loss-of-consciousness estimation system 100 of the embodiment, the first variation, and the second variation, the processing of step S113 does not necessarily need to be executed after step S112. The processing of step S113 may be executed at any timing as long as it is before the execution of the processing of step S114 and after the execution of the processing of step S109. For example, the processing of step S113 may be executed before the execution of the processing of step S111.

Note that the unit period is an example of one cycle.

The loss-of-consciousness estimation apparatuses 2 and 2a to 2e may be implemented using a plurality of information processing apparatuses that are communicably connected via a network. In this case, the functional units included in the loss-of-consciousness estimation apparatuses 2 and 2a to 2e may be mounted in a state of being distributed in a plurality of information processing apparatuses.

Note that all or some of the functions of the loss-of-consciousness estimation apparatuses 2 and 2a to 2e may be realized using hardware such as an ASIC (Application Specific Integrated Circuit), a PLD (Programmable Logic Device), and an FPGA (Field Programmable Gate Array). The program may be recorded on a computer-readable recording medium. The computer-readable recording medium is, for example, a flexible disk, a magneto-optical disk, a ROM, a portable medium such as a CD-ROM, or a storage apparatus such as a hard disk built in a computer system. The program may be transmitted via an electric communication line.

Although the embodiments of the present invention have been described in detail with reference to the drawings, the specific configuration is not limited to these embodiments, and includes designs and the like within a range that does not deviate from the gist of the present invention.

### REFERENCE SIGNS LIST

100, 100a, 100b, 100c, 100d, 100e Loss-of-consciousness estimation system
1 Biological sensor
2, 2a, 2b, 2c, 2d, 2e Loss-of-consciousness estimation apparatus
3 Respiration information acquisition sensor
20, 20a, 20b, 20c, 20d, 20e, 20f, 20g Control unit
21 Communication unit
22 Input unit
23 Storage unit
24 Output unit
210 Time series acquisition unit
220 Out-of-range data determination unit
221 First division unit
222 Distribution acquisition unit
223 Threshold region determination unit
224 Out-of-range data internal determination unit
230 Ventricular state estimation unit
231 Second division unit
232 Time integration unit
233 Peak period determination unit
234 Normal ventricular state estimation unit
235 Abnormal ventricular state estimation unit
240 Probability acquisition processing execution determination unit
250 Probability acquisition condition acquisition unit
260 Measurement reliability estimation unit
270 Loss-of-consciousness probability acquisition unit
280 Loss-of-consciousness determination unit
290 Output control unit
300 Recording unit
310 Grayout determination unit
320 Grayout condition determination unit
330 Probability determination condition candidate acquisition unit
340 Ventricular state estimation condition determination unit
350 Response acquisition unit
360 Learning update unit
370 Signal shaping unit
380 Signal modeling unit

1. A loss-of-consciousness estimation apparatus comprising:
a processor; and
a storage medium having computer program instructions stored thereon, when executed by the processor, perform to:
estimate whether or not a ventricular state of an estimation target is normal in a predetermined repetition cycle, based on a cerebral blood flow correlation amount time series, which is a time series of an amount correlated with a cerebral blood flow rate of the estimation target;
estimate reliability of the cerebral blood flow correlation amount time series; and
based on the reliability, the estimation result, and elapsed time after a probability acquisition start time, which is a time at which it is first determined that the ventricular state is not normal after an estimation start time, which is a time of starting the repetition cycle, acquire a loss-of-consciousness probability indicating a probability that the estimation target has already lost consciousness.

2. The loss-of-consciousness estimation apparatus according to claim 1, wherein the computer program instructions further perform to
determine whether or not the loss-of-consciousness probability exceeds a reference probability, which is a predetermined probability determined in advance.

3. The loss-of-consciousness estimation apparatus according to claim 1, wherein the computer program instructions further perform to if the reliability is a predetermined value or more and determines that the ventricular state of the estimation target is abnormal, acquires a loss-of-consciousness probability that is higher than a loss-of-consciousness probability acquired in one immediately-previous cycle, if the reliability is a predetermined value or more and determines that the ventricular state of the estimation target is normal, acquires a loss-of-consciousness probability that is lower than a loss-of-consciousness probability acquired in one immediately-previous cycle, and if the reliability is less than a predetermined value, acquires a loss-of-consciousness probability that is lower than a loss-of-consciousness probability acquired in one immediately-previous cycle.

4. The loss-of-consciousness estimation apparatus according to claim 1 wherein the computer program instructions further perform to determine whether or not the estimation target has grayed out based on the cerebral blood flow correlation amount time series.

5. A loss-of-consciousness estimation method comprising:
a ventricular state estimation step of estimating whether or not a ventricular state of an estimation target is normal in a predetermined repetition cycle, based on a cerebral blood flow correlation amount time series, which is a time series of an amount correlated with a cerebral blood flow rate of the estimation target;
a measurement reliability estimation step of estimating reliability of the cerebral blood flow correlation amount time series; and
a loss-of-consciousness probability acquisition step of, based on the reliability, the estimation result of the ventricular state estimation unit, and elapsed time after a probability acquisition start time, which is a time at which it is first determined in the ventricular state estimation step that the ventricular state is not normal after an estimation start time, which is a time of starting the repetition cycle, acquiring a loss-of-consciousness probability indicating a probability that the estimation target has already lost consciousness.

6. (canceled)
